(19) United States
(12) Patent Application Publication
(10) Pub. No.: US 2011/0045489 A1
(43) Pub. Date: Feb. 24, 2011

(54) Polymerases for Incorporating Modified Nucleotides

(75) Inventors: Andrew Gardner, Manchester, MA (US); Lucia Greenough, Ipswich, MA (US); William E. Jack, Wenham, MA (US)

Correspondence Address: HARRIET M. STRIMPEL, D. Phil., New England Biolabs, Inc., 240 COUNTY ROAD, IPSWICH, MA, 01938-2723, US

(73) Assignee: NEW ENGLAND BIOLABS, INC., Ipswich, MA (US)

(21) Appl. No.: 12/988,729

(22) PCT Filed: Apr. 20, 2009
(86) PCT No.: PCT/US09/41065

§ 371 Date: Oct. 20, 2010

Related U.S. Application Data
(60) Provisional application No. 61/046,987, filed on Apr. 22, 2008.

(51) Int. Cl. C12Q 1/68; C12N 9/12; C12P 19/34

(52) U.S. Cl. 435/6; 435/194; 435/91.5

(57) ABSTRACT

Compositions and methods are provided that relate to a recombinant protein with DNA polymerase activity in which one or more amino acids are mutated compared with the corresponding wild type protein. The recombinant protein is capable of incorporating one or more modified nucleotides into a nucleic acid substrate with a specific activity greater than 200.

### BACKGROUND

DNA polymerases have played a central role in the development of molecular biology. Their use is central in a wide range of laboratory protocols, including DNA sequencing (Sanger, et al., Proc. Natl. Acad. Sci., USA 74:5463-5467 (1977)), strand displacement amplification (SDA; Walker, et al., Proc. Natl. Acad. Sci., USA 89:392-396 (1992)), probe-labeling, site-directed mutagenesis, polymerase chain reaction (PCR) (Saiki, et al., Science, 230:1350-1354 (1985)) and cloning. These applications depend critically on the ability of polymerases to faithfully replicate DNA.

A number of applications require polymerases that are able to incorporate modified nucleotides. For example, chain terminator DNA sequencing utilizes incorporation of a chain-terminating nucleotide, most often a ddNTP, to deduce the pattern of bases in a sequencing sample (Sanger, et al., Proc. Natl. Acad. Sci., USA 74:5463-5467 (1977)). Additional applications rely on incorporation of nucleotides with modified bases, often to aid in detection of the polymerized product. One such application is the incorporation of nucleotides with fluorescent bases, allowing analysis of the products of chain-terminating DNA sequencing reactions (Prober, et al., Science 238:336-341 (1987)). Modifications of this method have also been described that focus on single nucleotide loci, allowing detection of single nucleotide polymorphisms (Ellison, et al., Biotechniques 17:742-753 (1994); Chen and Kwok, Nucleic Acids Res. 15:347-353 (1997); Chen et al., Genome Research 9:492-498 (1999)). Such single-base detection methods have been instructive in genetic testing and analysis.

Sequencing-by-synthesis methods (Metzker Genome Res 15(12): 1767-76 (2005)) have emerged as an important technology that have enabled DNA sequencing on a massive scale while reducing costs compared to conventional methods. Sequencing-by-synthesis methods rely on incorporation and detection of “reversible terminators” by a DNA polymerase. Reversible 3′-modified nucleotide terminators can be used in massively parallel sequencing-by-synthesis methods. Inadequate terminal incorporation of 3′-modified nucleotides by naturally occurring DNA polymerases is a limiting factor in the success of these methods. It would be desirable to create a novel DNA polymerase that provides improved efficiency of incorporation of 3′-modified nucleotides to overcome this limitation.

Another difficulty encountered in the methods stated above arises from the inherent fidelity of naturally occurring DNA polymerases. This results in a bias against modified nucleotide incorporation that can sometimes be overcome by increasing the concentration of modified nucleotides in the reaction mixture. Even then, incorporation may be incomplete and non-uniform, limiting the sensitivity of detection and producing patterns that may not accurately reflect the actual nucleotide sequence being replicated. This complicates determinations such as DNA sequence analysis.

Accordingly, there is a need in the art for DNA polymerases that more readily incorporate modified nucleotides. Since a number of methods require a step in which the DNA is denatured at temperatures up to 95° C., thermostable DNA polymerases are preferable. One thermostable enzyme that has been extensively used is Taq DNA polymerase, along with a variety of engineered versions of this enzyme. Extensive studies have characterized the ability of this enzyme to incorporate nucleotides that act as terminators (e.g., ddNTPs) and nucleotides with modified bases (e.g., dye-labeled). Such modifications can affect polymerization. For example, terminator DNA sequencing reactions with the F667Y version of Taq DNA polymerase (also know by the trade name Thermosequenase™, USB, Inc., Cleveland, Ohio) show “ . . . less uniform peak height patterns when compared to primer chemistry profiles, suggesting that the dyes and/or their linker arms affect enzyme selectivity.” (Brandis, Nucleic Acids Res. 27:1912-1918 (1999)).

Taq DNA polymerase is a Family A DNA polymerase. Amino acid similarities allow the classification of most DNA polymerases into three Families, A, B and C, according to similarities with Escherichia coli polymerases I, II and III, respectively (Ito and Braithwaite, Nucleic Acids Res. 19:4045-4057 (1991); Heringa and Argos, The Evolutionary Biology of Viruses, Morse, S. S., ed., pp. 87-103, Raven Press, New York (1992)).

Family B polymerases include thermostable polymerases from thermophilic archaea. One such example is Vent® (New England Biolabs (NEB), Inc., Ipswich, Mass.) DNA polymerase, originally isolated from Thermococcus litoralis (Perler, et al., Proc. Natl. Acad. Sci. USA 89:5577-5581 (1992)). Vent® DNA polymerase has a relatively high Kₘ for nucleotides (Kong, et al., J. Biol. Chem. 268:1965-1975 (1993)), and functions poorly in incorporating dye-substituted terminators in DNA sequencing reactions (“CircumVent™: Questions and Answers,” The NEB Transcript, September 1992, p. 12-13). Incorporation of unsubstituted dideoxynucleoside triphosphate (ddNTP) terminators is also inefficient with this enzyme (Gardner and Jack, Nucleic Acids Res. 27:2545-2553 (1999)). Thus, Vent® DNA polymerase does not appear to be a promising candidate for applications requiring incorporation of modified nucleotides.

Archaeon DNA polymerase mutants have been described that somewhat increase the incorporation efficiency of specific classes of chain terminators, namely ddNTPs and 3′-dNTPs. 9° N exo-Y409A/A485L (Therminator™ II, NEB, Inc., Ipswich, Mass.) has been used in massive parallel sequencing with 3′-modified reversible terminators Seo et al. J Org Chem 68(2): 609-12 (2003)). However, the incorporation of the 3′ modified reversible terminator by this polymerase mutant was relatively inefficient and required long incubation times and high concentrations of 3′-modified nucleotide reversible terminators to complete the reaction.

Because DNA polymerases discriminate against nucleotide analogs such as ddNTPs and rNTPs resulting in reduced binding affinity and slowed rates of catalysis (Gardner et al. J Biol Chem 279(12): 11834-42 (2004)), it has proved extremely challenging to engineer DNA polymerases that will incorporate non-natural nucleotide analogs with fidelity while maintaining high reaction efficiency. Canard et al. (Proc Natl Acad Sci USA 92(24): 10859-63 (1995)) demonstrated incorporation of 3′-esterified nucleotides by DNA polymerases but noted that incorporation of these modified nucleotides was inefficient. In addition, DNA polymerases could use the 3′-esterified linkage as a template to add a subsequent deoxynucleotide triphosphosphate (dNTP) on the 3′ end (Canard et al. Proc Natl Acad Sci USA 92(24): 10859-63 (1995)). In light of the above, it would be desirable to design a DNA polymerase with a higher efficiency of 3′-modified nucleotide incorporation.

### SUMMARY

In an embodiment of the invention, a recombinant protein with DNA polymerase activity is described that may be characterized as containing an amino acid sequence that has at least 90% amino acid sequence identity with SEQ ID NO:1. One or more amino acids in the recombinant protein are mutated compared with the corresponding wild type protein. The mutation for example may be located in SEQ ID NO:1 or in Region III. The recombinant protein is capable of (i) incorporating one or more nucleotides into a nucleic acid substrate with a specific activity greater than 200, more specifically a specific activity of greater than 1000, more specifically a specific activity of greater than 5000; and (ii) incorporating one or more modified nucleotides into the nucleic acid substrate with at least two fold greater efficiency than for corresponding wild type DNA polymerase.

Examples of the recombinant protein include: a 9° N archael polymerase, and the mutated amino acids comprising D141A and E143A and an additional mutation selected from the group consisting of: P410V; S411T; L408S/Y409A/P410V; L408P/Y409A/S411T; P410R/S411T; L408S/Y409A/P410V/S411T; L408P/Y409A/P410V/S411T; N491L/Y494S; N491V/Y494H; R406S/L408R; R406L/L408E; R406T/L408R; R406V/L408R; R406T/L408E; R406V/L408R; R406E/L408G; R406P/L408G; Y409A/R406V; Y409A/R406S/L408K; Y409A/R406S/L408R; Y409A/R406T/L408K; Y409A/R406T/L408R; Y409A/R406H/L408G; Y409A/R406Y/L408G; Y409A/R406L/L408G; Y409A/R406P/L408C; Y409A/R406S/L408I; Y409A/R406V/L408Y; Y409A/R406V/A485L; N491L/Y494S; N491V/Y494H; Y409A/R406 (nucleophilic amino acid)/L408 (basic amino acid); Y409A/R406 (hydrophobic amino acid)/L408 (small amino acid); Y409A/R406/L408 (hydrophobic amino acid)/L408 (small amino acid); P410V/A485L; L408S/P410V/A485L; Y409A/S411T/A485L; L408S/Y409A/P410V/A485L; L408P/Y409A/S411T/A485L; Y409A/P410R/S411T/A485L; L408S/Y409A/P410V/S411T/A485L; L408P/Y409A/P410V/S411T/A485L; N491L/Y494S/A485L; N491V/Y494H/A485L; R406S/L408R/A485L; R406L/L408E/A485L; R406T/L408R/A485L; R406V/L408R/A485L; R406T/L408E/A485L; R406V/L408R/A485L; R406E/L408G/A485L; R406P/L408G/A485L; Y409A/R406S/L408K/A485L; Y409A/R406S/L408R/A485L; Y409A/R406T/L408K/A485L; Y409A/R406T/L408R/A485L; Y409A/R406H/L408G/A485L; Y409A/R406Y/L408G/A485L; Y409A/R406L/L408G/A485L; Y409A/R406P/L408C/A485L; Y409A/R406S/L408I/A485L; Y409A/R406V/L408Y/A485L.

Additional examples include a recombinant protein from Methanococcus maripolludis archael DNA polymerase, where the mutated amino acids are selected from D153A/E155A/L417S/P419V and D153A/E155A/L417P/Y418A/S420T.

Examples of modified nucleotides include nucleotides that are selected from 3′ terminators and 3′ reversible terminators, for example, according to the following chemical structure, when N is a nucleoside, and the R group on the 3′ position of the ribose may be larger than a hydroxyl group. In particular, the R group may be substituted by the groups as listed here

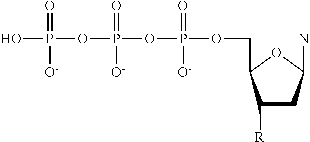

R═—H, —SH, —N₃, —F, —Cl, -azidomethyl, —NH₂, -anthranyloyl-fluothioureido, -chain, -amd, —O-allyl, —O-aminoallyl, —O-azidomethyl, —O-methyl, —O-phophate, —O-diphophate, —O-(2-nitrobenzyl), —O—[N6(anthranyl)amidohex

Additionally, modified nucleotides may be selected from the group consisting of:

2′-deoxy-3′-anthranyloyl-dNTPs (3′-ant-dNTPs) 3′-{N3-[3-carboxylato-4-(3-oxido-6-oxo-6H-xanthen-9-yl)phenyl]thioureido}-3′-deoxythymidine 5′-triphosphate (3′-fluothioureido-dTTP), 3′-deoxy-3′-(N-methylanthranyloylamino)thymidine 5′-triphosphate (3′-amd-dTTP), 3′-O—[N6(N-methylanthranyl)amidohexanoyl]-dGTP (3-chain-dGTP), and 3′-O—[N6(anthranyl)amidohex (3′-chain-dATP).

Examples of modified nucleotides include labelled modified nucleotides including fluorescent labels.

In an embodiment of the invention, a method is provided for incorporating modified nucleotides into a nucleic acid by reacting a nucleic acid with a recombinant protein as described above and at least one modified nucleotide.

In an additional embodiment of the invention, a kit is provided that contains a recombinant protein as described above and instructions for use. The kit may further include a modified nucleotide.

In a further embodiment of the invention, a method is provided of screening for a recombinant protein as described above wherein the method includes: (a) determining the size of a substrate after addition of a modified nucleotide during a polymerization reaction; and (b) measuring at least one of an increase in chain terminator incorporation, and a decrease in average reaction product size, to determine efficiency of incorporation by the composition.

### BRIEF DESCRIPTION OF THE DRAWINGS

FIG. 1A-1D show titration assays for chain terminator incorporation efficiency. The relative efficiency of incorporation of modified nucleotide terminators was assessed using the titration assay described in Gardner and Jack Nucleic Acids Research 30: 605-613 (2002). A dye-labeled oligonucleotide primer 5′-AGTGAATTCG AGCTCGGTAC CCGGGGATCC TCTAGAGTCG ACCTGCAGGC-3′ was annealed to a single-stranded M13mp18 DNA template (Accession No. X02513) and extended by a DNA polymerase in the presence of various ratios of 3′-azido-ddCTP:dNTP (10:1, 2.5:1, 1:2.5) or dNTP (“dN” in the figure) alone in 1×ThermoPol buffer (20 mM Tris-HCl, 10 mM (NH₄)₂SO₄,10 mM KCl, 2 mM MgSO₄, 0.1% Triton X-100, pH 8.8 @ 25° C.). Reactions were incubated and termination products were resolved on 20% denaturing polyacrylamide gel electrophoresis (PAGE).

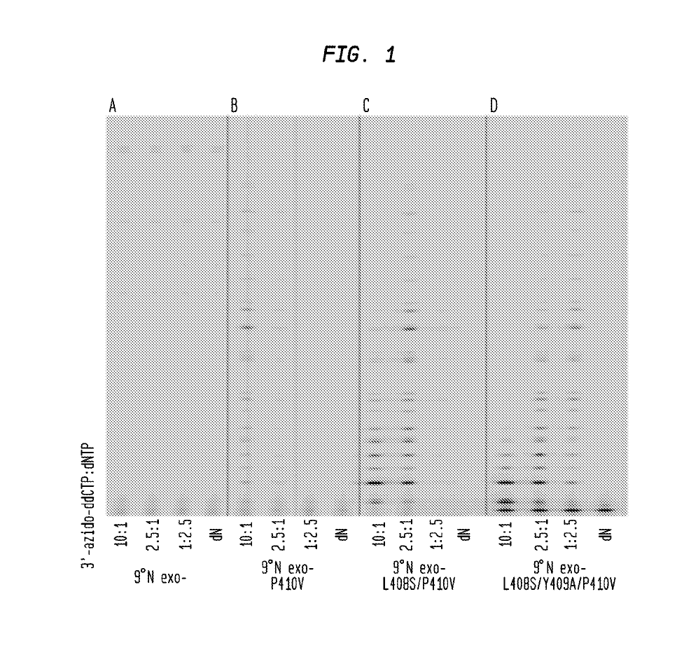

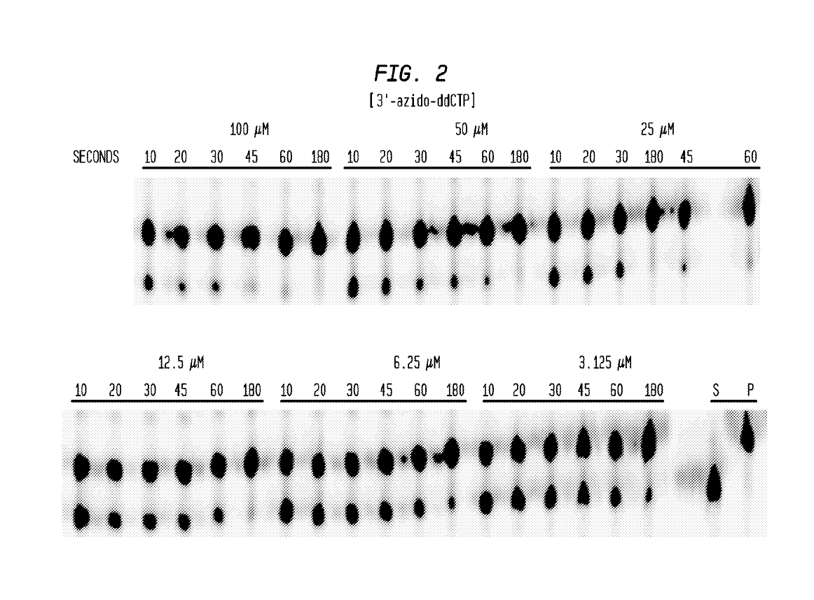

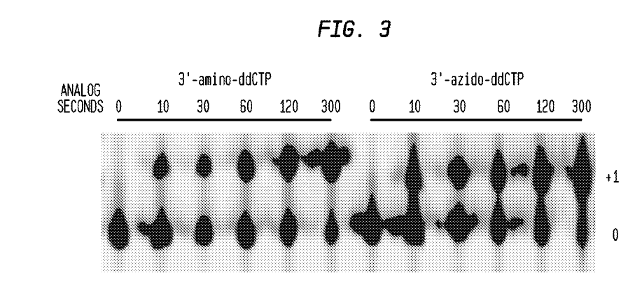

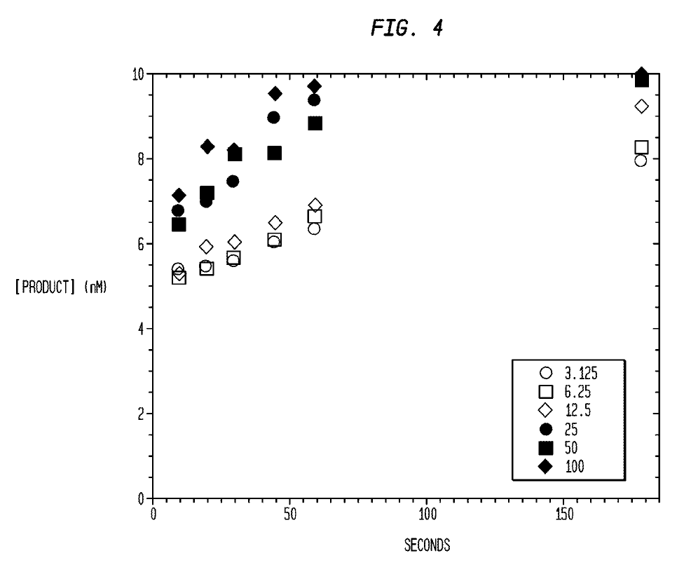

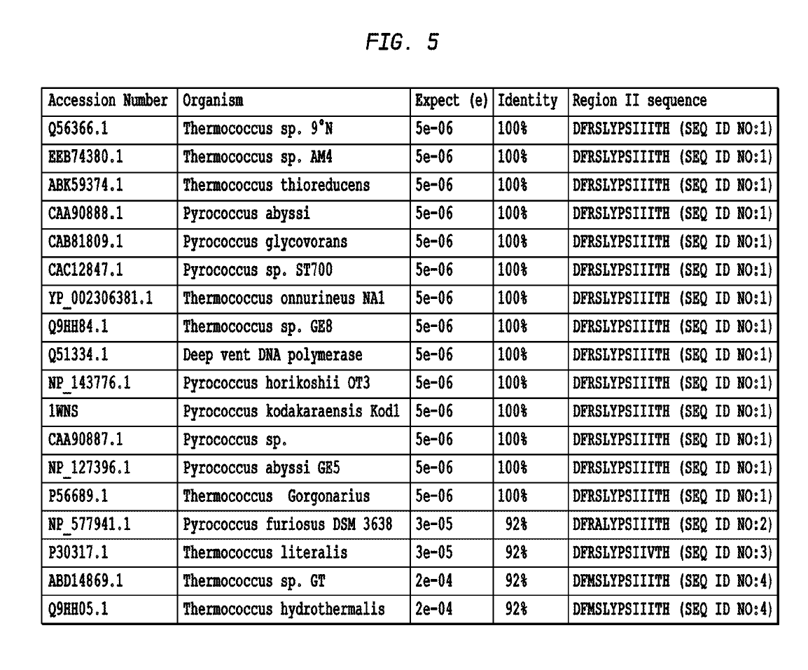

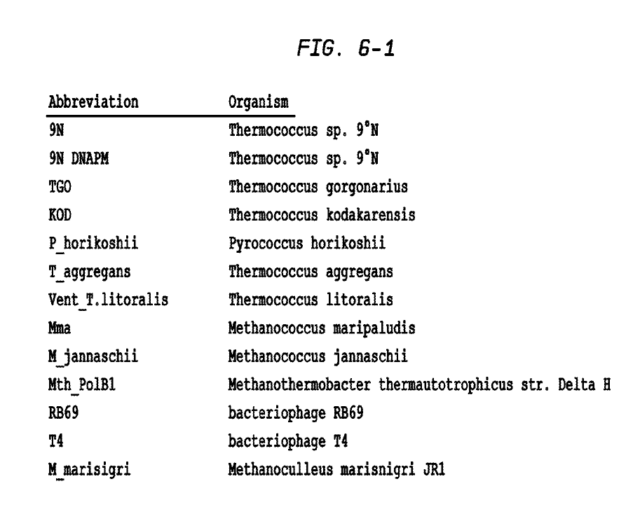

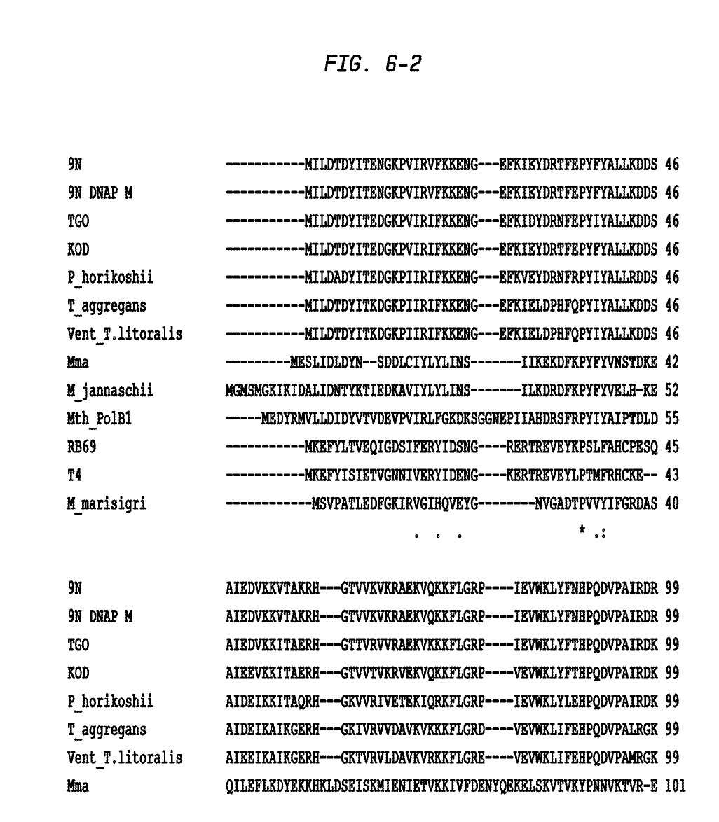

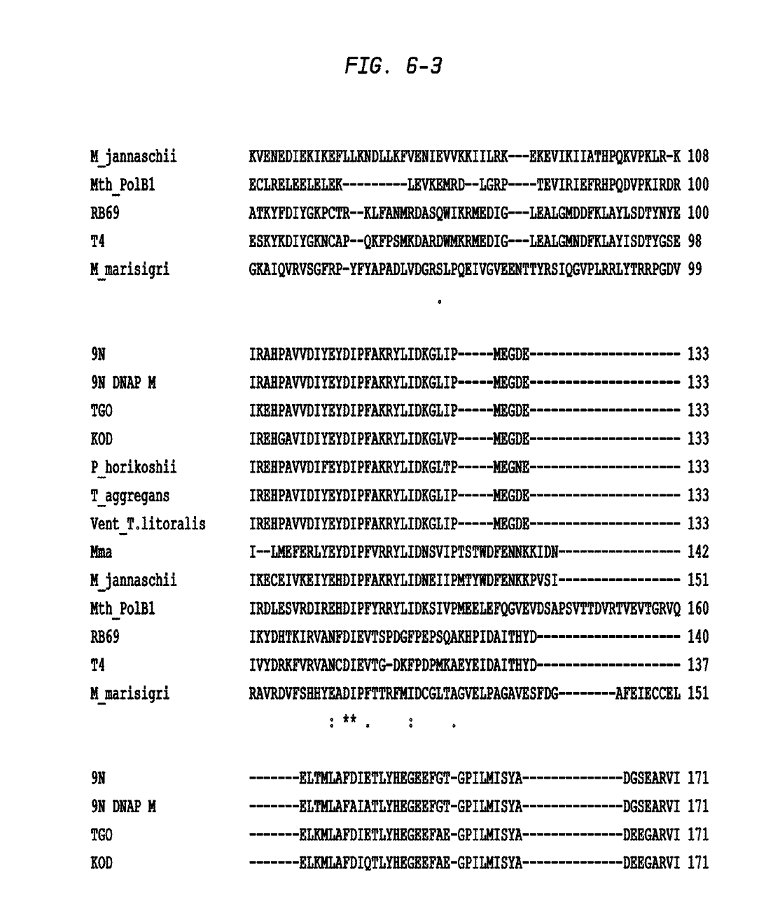

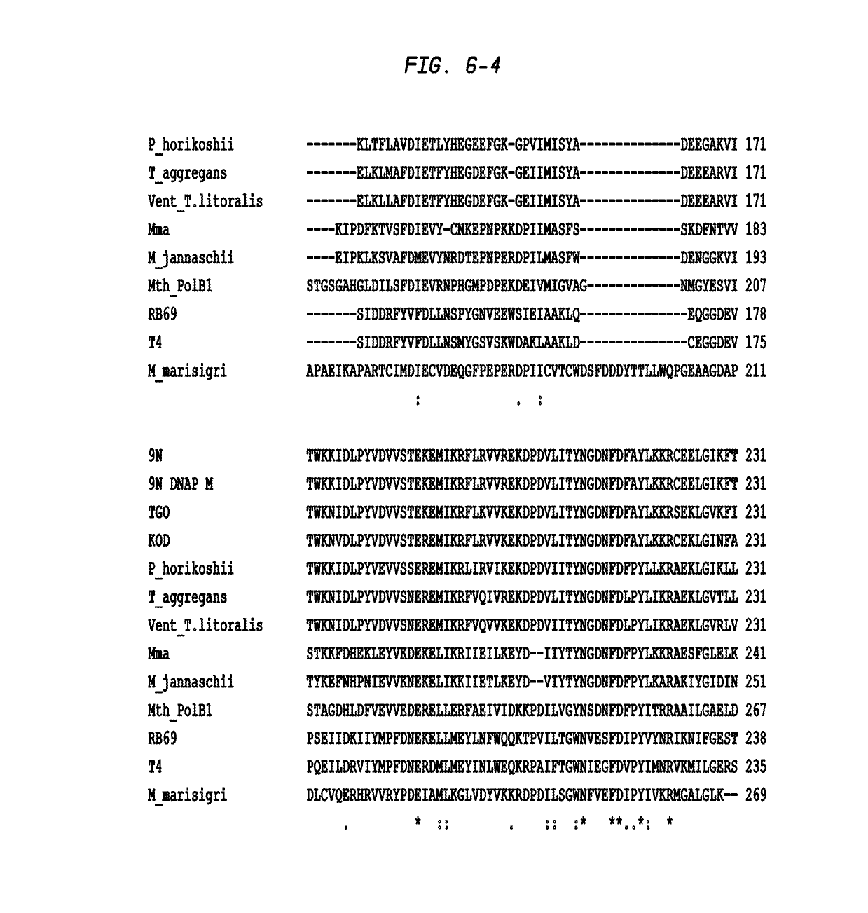

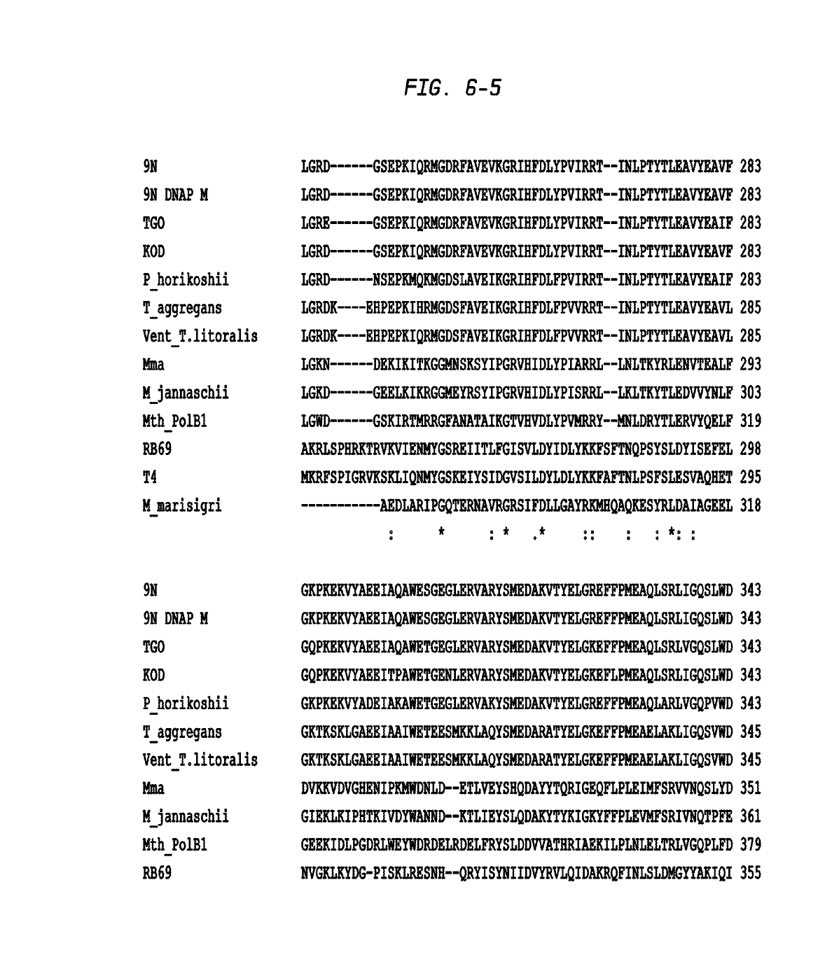

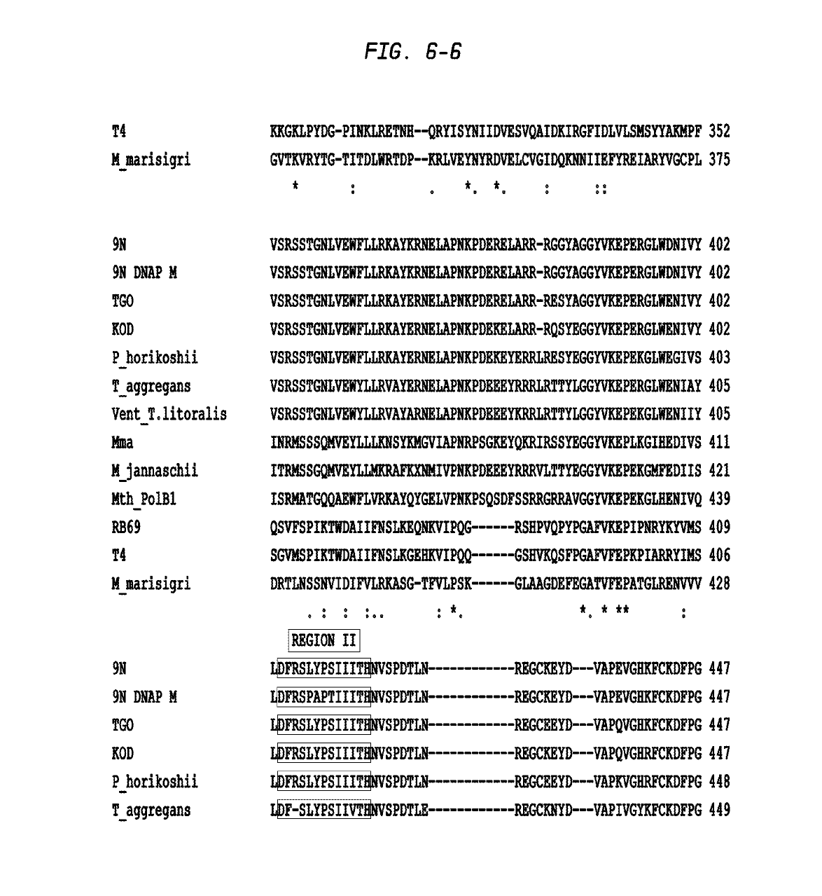

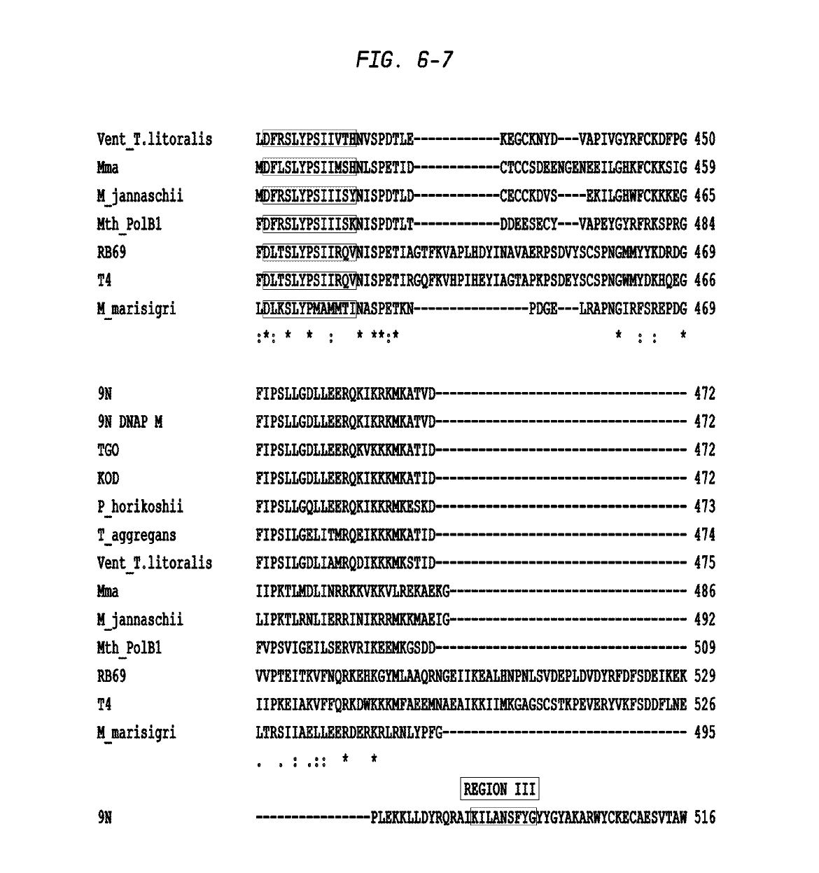

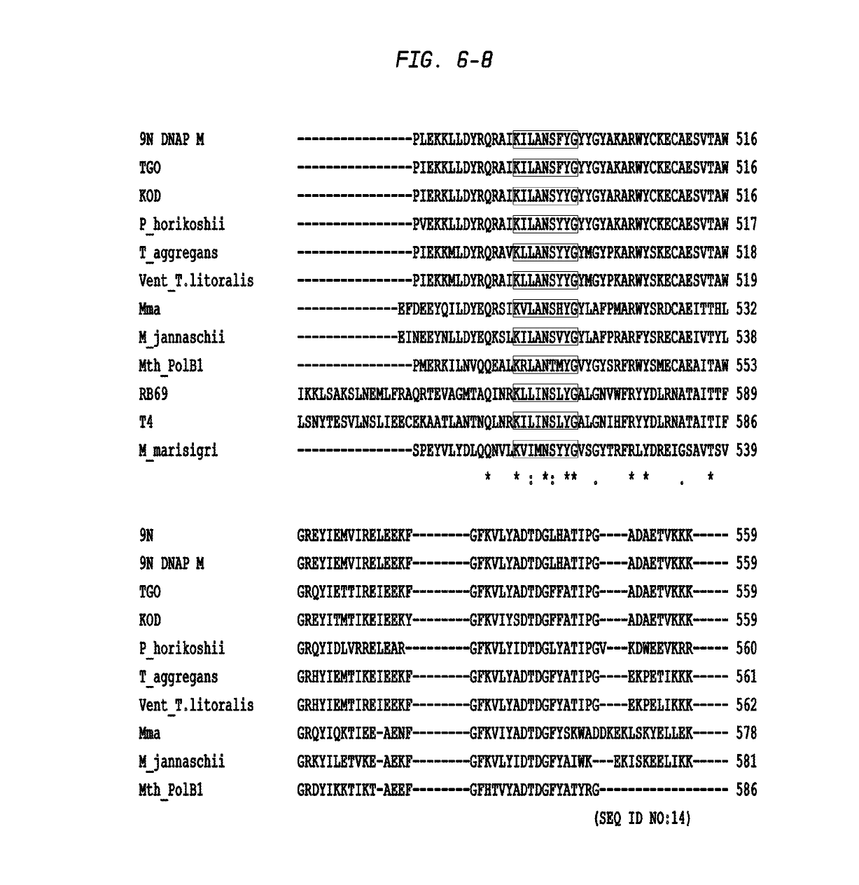

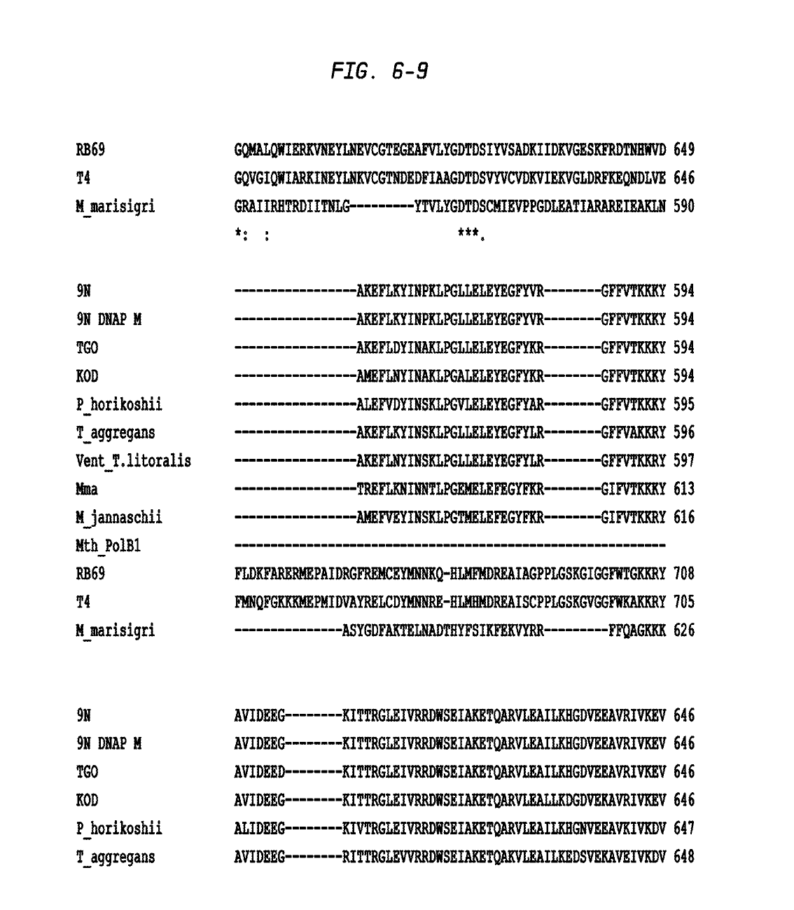

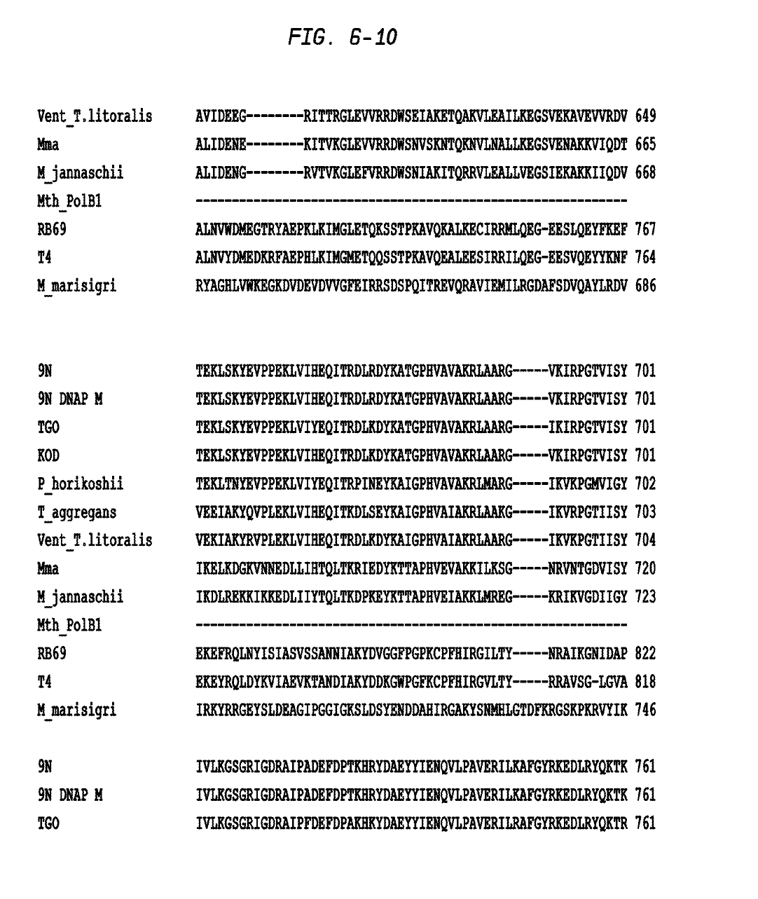

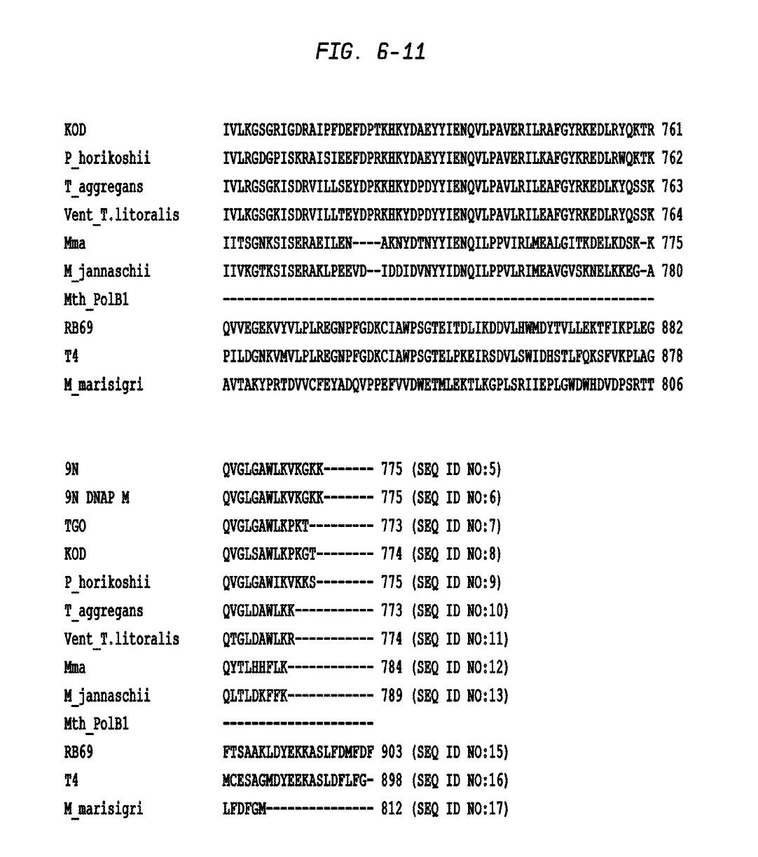

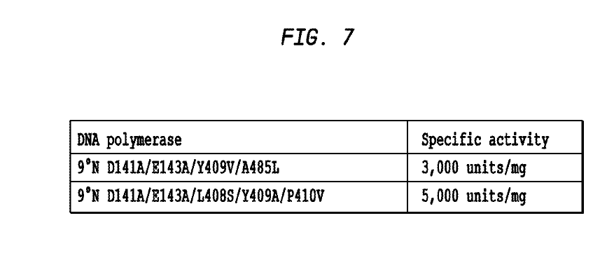

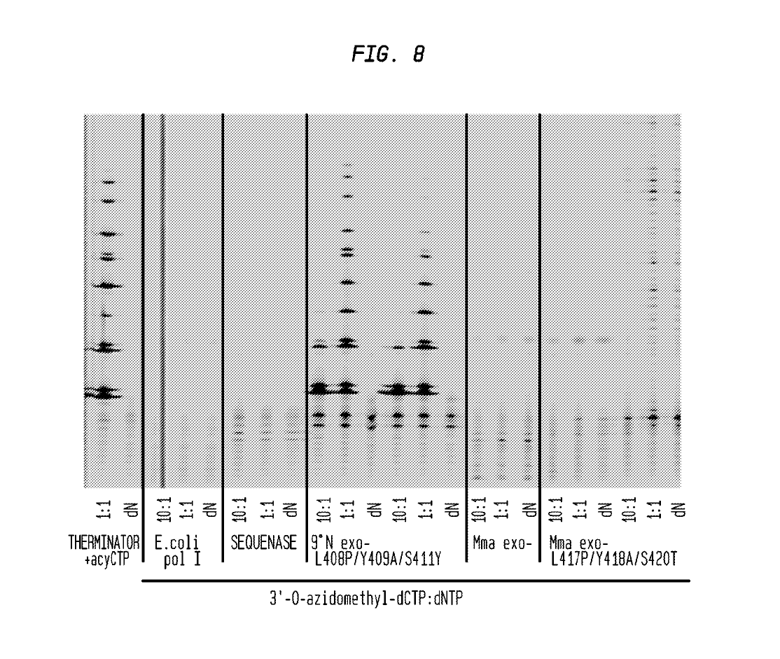

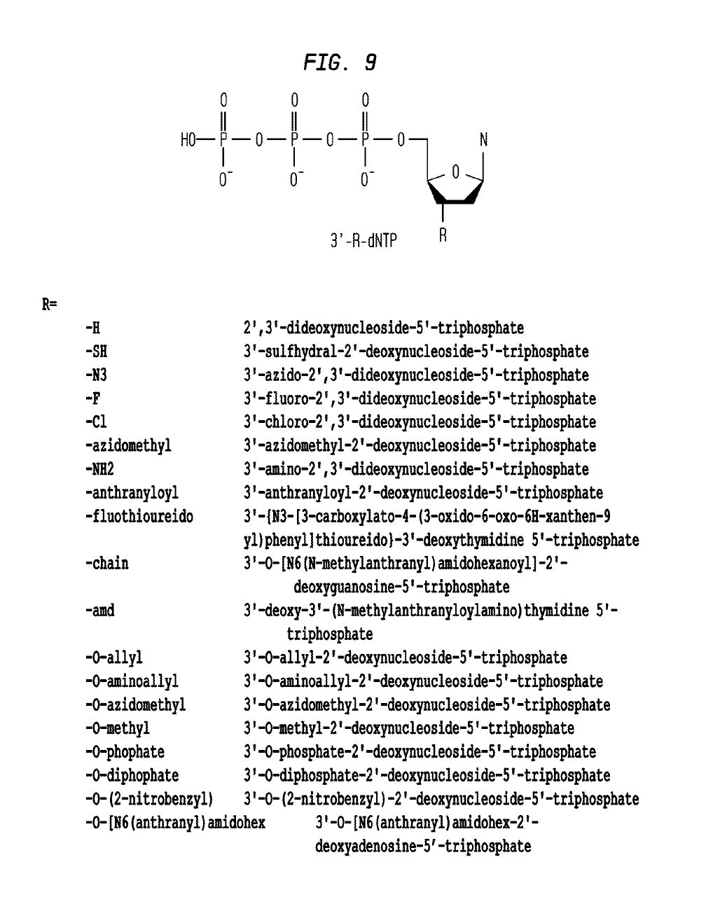

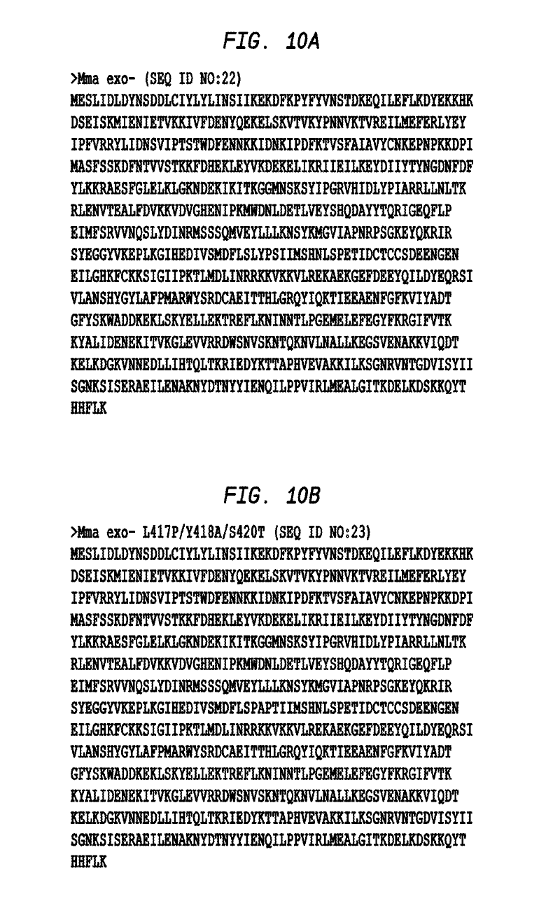

Bands correspond to DNA with an added 3′-azido-ddCMP terminator. This showed that DNA polymerase mutants that increased incorporation efficiency required less 3′-azido-ddCTP to produce termination fragments than did the corresponding non-mutated DNA polymerase. The results show that non-mutated DNA polymerases failed to produce termination fragments even at a 10:1 ratio of 3′-azido-ddCTP terminator:dNTP. FIG. 1A is 9° N exo-. FIG. 1B is 9° N exo-P410V. FIG. 1C is 9° N exo-L408S/P410V and FIG. 1D is 9° N exo-L408S/Y409A/P410V. 9° N exo-L408S/P410V DNA polymerase (FIG. 1C) incorporated 3′-azido-ddCTP at least 2-fold more efficiently than wild-type 9° N exo-DNA polymerase (FIG. 1A) and at least 4-fold more efficiently than 9° N exo-P410V DNA polymerase (FIG. 1B). 9° N exo-L408S/Y409A/P410V DNA polymerase (FIG. 1D) incorporated 3′-azido-ddCTP at least 4-fold more efficiently than 9° N exo-L408S/P410V DNA polymerase (FIG. 1C).

FIG. 2 shows a kinetic analysis of 3′-azido-ddCTP incorporation by 9° N exo-L408S/Y409A/P410V DNA polymerase. An IR800-dye-labeled synthetic primer 5′-AGTGAATTCG AGCTCGGTAC CCGGGGATCC TCTAGAGTCG ACCTGCAGGC-3′ (SEQ ID NO:18) was annealed to a template 3′-TCACTTAAGC TCGAGCCATG GGCCCCTAGG AGATCTCAGC TGGACGTCCG GATCCTATAC TAATCCC-5′ (SEQ ID NO:19) and used as a substrate to measure rates of 3′-azido-ddCTP incorporation. Incorporation of varying concentrations of 3′-azido-ddCTP (100, 50, 25, 12.5, 6.25, and 3.125 μM) by 9° N exo-L408S/Y409A/P410V DNA polymerase was monitored over a three-minute time course. Reaction aliquots were sampled at 10, 20, 30, 45, 60, and 180 seconds and analyzed by 20% denaturing PAGE. “S” is the unextended primer and “P” is the fully extended product corresponding to 3′-azido-ddCMP addition at the +1 position. The gel shows 2 bands, the larger band corresponding to the extension of the primer by the terminator. The extent of the incorporation was shown to be dependent on the concentration of modified nucleotide such that the greater the concentration of the primer, the faster the reaction.

FIG. 3 shows a kinetic analysis of 3′-amino-ddCTP and 3′-azido-ddCTP incorporation by 9° N exo-L408S/Y409A/P410V DNA polymerase. An IR800-dye-labeled synthetic primer 5′-AGTGAATTCG AGCTCGGTAC CCGGGGATCC TCTAGAGTCG ACCTGCAGGC-3′ (SEQ ID NO:18) was annealed to a template 3′-TCACTTAAGC TCGAGCCATG GGCCCCTAGG AGATCTCAGC TGGACGTCCG GATCCTATAC TAATCCC-5′ (SEQ ID NO:19) and used as a substrate. Reaction aliquots were sampled at 10, 30, 60, 120 and 300 seconds and analyzed by 20% denaturing PAGE. Unextended primer (0) and fully extended product (+1) are indicated. The gel shows that 10 μM of each of the modified nucleotides is sufficient for efficient incorporation by 9° N exo-L408S/Y409A/P410V DNA polymerase.

FIG. 4 shows a time course plot of 3′-azido-ddCTP incorporation by 9° N exo-L408S/Y409A/P410V DNA polymerase. Linear rates of 3′-azido-ddCTP incorporation derived from these curves are 100 μM: 0.05 s⁻¹; 50 μM: 0.05 s⁻¹; 25 μM: 0.05s⁻¹; 12.5 μM: 0.03s⁻¹; 6.25 μM: 0.03s⁻¹; 3.125 μM: 0.02 s⁻¹.

FIG. 5 shows a sequence similarity search for Family B DNA polymerase Region II. Region II in 9° N DNA polymerase (DFRSLYPSIIITH) (SEQ ID NO:1) was used to query Genbank for highly similar sequences (>90% identity; e-values less that 0.003) using BLAST (Altschul et al. Nucleic Acids Res 25(17): 3389-402 (1997)).

FIGS. 6-1 to 6-11 show the results of a Clustal W sequence in which Family B DNA polymerases from archaea and bacteriophage were aligned. Family B DNA polymerases from Thermococcus sp. 9° N (9N), Thermococcus sp. 9° N mutant (9N DNAP), Thermococcus gorgonarius (TGO), Thermococcus kodakarensis (KOD), Pyrococcus horikoshii (P_horikoshii), Thermococcus aggregans (T_aggregans), Thermococcus litoralis (Vent_T. litoralis), Methanococcus maripaludis (Mma), Methanococcus jannaschii (M_jannaschii), Methanothermobacter thermautotrophicus str. Delta H (Mth_PolB1), bacteriophage RB69 (RB69), bacteriophage T4 (T4), and Methanoculleus marisnigri JR1 (M_marisigri) were aligned using software Clustal W 2.0 (http://www.ebi.ac.uk/Tools/clustalw2/index.html). Amino acids in Region II and Region III are highlighted in boxes. Conserved amino acids are noted with an asterisk and similar amino acids by a dot (.) or colon (:).

FIG. 7 shows specific activities for 9° N D141A/E143A/L408S/Y409A/P410V and 9° N D141A/E143A/Y409V/A485L DNA polymerase dNTP incorporation.

FIG. 8 shows the results of extending a 5′-dye-labeled oligonucleotide primer 5′-AGTGAATTCG AGCTCGGTAC CCGGGGATCC TCTAGAGTCG ACCTGCAGGC-3′ (SEQ ID NO:18) annealed to a single-stranded M13mp18 DNA template (Accession No. X02513) by various DNA polymerases and mutants. The polymerases used were an E. coli DNA polymerase I (pol I) (NEB, Inc., Ipswich, Mass.), Sequenase™ (USB, Inc., Cleveland, Ohio), 9° N exo-L408P/Y409A/S411T (SEQ ID NO:6), Mma exo-(SEQ ID NO:22) (FIG. 10A), or Mma exo-L417P/Y418A/S420T (SEQ ID NO:23) (FIG. 10B) in the presence of various ratios with 10:1 or 1:1 3′-O-azidomethyl-dCTP:dNTP. Reactions were also performed in the absence of terminators to ensure that synthesis by each DNA polymerase was sufficient to extend primers without premature termination (“dNTP” lanes). Reactions with Therminator™ (NEB, Inc., Ipswich, Mass.) and 9° N exo-L408P/Y409A/S411T DNA polymerase (SEQ ID NO:6) were incubated at 72° C. for 30 minutes. Reactions with E. coli Polymerase I (pol I) (NEB, Inc., Ipswich, Mass.), Sequenase™, (USB, Inc., Cleveland, Ohio), Mma exo- and Mma exo-L417P/Y418A/S420T were incubated at 37° C. for 30 minutes. Each band corresponds to a DNA fragment terminated by a 3′-azido-dCMP. Using a titration assay as described in Gardner and Jack (2002), the relative 3′-azido-dCTP incorporation efficiency was determined for a series of DNA polymerases. The incorporation efficiency for 9° N exo-L408P/Y409A/S411T was greater than Mma exo-L417P/Y418A/S420T, which was greater than Mma exo-, which was greater than Sequenase™ (USB, Inc., Cleveland, Ohio), which was greater than E. coli DNA polymerase I (pol I) (NEB, Inc., Ipswich, Mass.).

FIG. 9 shows examples of nucleotide terminators modified at the 3′ position (R). “N” can be adenine, cytosine, guanosine or thymine.

FIG. 10A is the amino acid sequence (SEQ ID NO:22) of an exonuclease minus DNA polymerase from Mma exo-created by site-directed mutagenesis to change D141A and E143A. Mma exo- was used to evaluate incorporation of 3′-O-azidomethyl-dCTP in FIG. 8 and Example 4.

FIG. 10B is the amino acid sequence (SEQ ID NO:23) of an exonuclease minus mutant DNA polymerase from Mma exo-created by site-directed mutagenesis to change D141A and E143A and L417P/Y418A/S420T. Mma exo-L417P/Y418A/S420T was used to evaluate incorporation of 3′-O-azidomethyl-dCTP in FIG. 8 and Example 4.

### DETAILED DESCRIPTION OF THE EMBODIMENTS

Embodiments of the present invention describe modified Family B archaeon DNA polymerases and related codon-substituted mutants capable of incorporating selected modified nucleotides into nucleic acids with improved efficiency. Examples include: Vent® DNA polymerase (Kong, et al., J. Biol. Chem. 268:1965-1975 (1993); and U.S. Pat. Nos. 5,500,363, 5,834,285, and 5,352,778); Pyrococcus furiosus (Pfu) DNA polymerase (U.S. Pat. Nos. 5,489,523 and 5,827,716); Thermococcus barossii (Tba) DNA polymerase (U.S. Pat. No. 5,882,904); and 9° N7 DNA polymerase (Southworth et al. Proc. Natl. Acad. Sci. USA 93: 5281-5285 (1996)).

Some of the above polymerases have 3-5′ exonuclease activity. One function of this activity is “proofreading,” wherein the polymerase can remove 3′ nucleotides before proceeding with polymerization. Incorrectly base-paired nucleotides, or aberrant nucleotides are preferentially removed by this activity, thus increasing the fidelity of replication (Kornberg, DNA Replication p. 127 (1980)). Modified nucleotides might reasonably be expected to be identified as “aberrant,” and, even if incorporated, be subject to removal by this activity. To avoid this possibility, mutants can be created that lack or have diminished exonuclease activity. Such mutants include Family B archeal DNA polymerases with ≧90% identity to 9° N DNA polymerase and with ≧30% and <90% identity to 9° N DNA polymerase as follows:

Family B archaeal DNA polymerases with ≧90% identity to 9° N DNA polymerase can be obtained from host cells such as: Pyrococcus kodakaraensis, Pyrococcus furiosus, Pyrococcus woesei, Pyrococcus glycovorans, Pyrococcus abyssi, Pyrococcus sp. GB-D, Pyrococcus sp. ST700, Pyrococcus horikoshii OT3, Thermococcus litoralis, Thermococcus gorgonarius, Thermococcus sp AM4, Thermococcus sp. GE8, Thermococcus thioreducens, Thermococcus onnurineus NA1, Thermococcus sp. GT, Thermococcus zilligii, Thermococcus hydrothermalis, Thermococcus fumicolans, Thermococcus barophilus MP, and Thermococcus sp. TY.

Family B archaeal DNA polymerases with ≧30% and <90% identity to 9° N DNA polymerase can be obtained from host cells such as: Aciduliprofundum boonei, Aeropyrum pernix, Archaeoglobus fulgidus, Caldivirga maquilingensis, Candidatus korarchaeum cryptofilum, Desulfurococcus kamchalkensis, Hyperthermus butylicus, Ignicoccus hospitalis KIN4/I, Methanosphaera stadtmanae, Metallosphaera sedula, Methanobrevibacter smithii, Methanocaldococcus jannaschii, Methanococcoides burtonii, Methanococcus maripaludis, Methanococcus vannielii, Methanococcus aeolicus Nankai-3, Methanococcus voltae A3, Methanopyrus kandleri AV19, Methanosaeta thermophila, Methanosarcina mazei, Methanosarcina acetivorans, Methanothermobacter thermoautotrophicus, Pyrobaculum calidifontis, Pyrobaculum aerophilum, Pyrobaculum arsenaticum, Pyrobaculum islandicum, Pyrodictium occultum, uncultured methanogenic archaeon (YP_{—}687422.1), Staphylothermus marinus, Sulfolobus tokodaii, Sulfophobococcus zilligii, Sulfurisphaera ohwakuensis, Thermodesulfovibrio yellowstonii, Thermofilum pendens Hrk 5, Thermoproteus neutrophilus and uncultured euryarchaeote Alv-FOS1.

Reversible terminators for use in embodiments of the invention contain a protecting group attached to the 3′-OH ribose position that terminates DNA synthesis. Removal of the protecting group restores the unblocked natural nucleotide substrate, allowing subsequent addition of reversible terminators.

Examples of reversible terminators include 3′-O-azidomethyl-2′-deoxynucleoside-5′-triphosphate and 3′-O-(2-nitrobenzyl)-2′-deoxynucleoside-5′-triphosphate (Ruparel et al. Proc Natl Acad Sci USA 102(17): 5932-7 (2005); Wu et al. Proc Natl Acad Sci USA 104(42): 16462-7 (2007) and Guo et al. Proc Natl Acad Sci USA 105(27): 9145-50 (2008)). Other examples of modified nucleotides suitable for incorporation into nucleic acids by archaeal DNA polymerases include: 3′-modified nucleotide analogs in which the 3′-position of the deoxyribose in the nucleotide analogue can be: azidomethyl; O-azidomethyl, azido, sulfhydral, amino, fluorine, chlorine, —O-methyl, O-phosphate, O-diphosphate, aminoallyl, O-aminoallyl, hydrogen (Bi et al. J Am Chem Soc 128(8): 2542-3 (2006); Kim et al. Nat Rev Genet 4(12): 1001-8 (2006); Turcatti et al. Nucleic Acids Res 36(4): e25 (2008); and Foldesi et al. Nucleosides Nucleotides Nucleic Acids 26(3): 271-5 (2007)). See also catalogs of Trilink Biotechnologies (San Diego, Calif.) and Jena Biosciences (Jena, Germany).

Other examples include, 2′-deoxy-3′-anthranyloyl-dNTPs (3′-ant-dNTPs) 3′-{N3-[3-carboxylato-4-(3-oxido-6-oxo-6H-xanthen-9-yl)phenyl]thioureido}-3′-deoxythymidine 5′-triphosphate (3′-fluothioureido-dTTP), 3′-deoxy-3′-(N-methylanthranyloylamino)thymidine 5′-triphosphate (3′-amd-dTTP), 3′-O—[N6(N-methylanthranyl)amidohexanoyl]-dGTP (3-chain-dGTP), and 3′-O—[N6(anthranyl)amidohex (3′-chain-dATP) Canard et al. Proc Natl Acad Sci USA 92(24): 10859-63 (1995)). In addition, reversible terminators can be conjugated with dyes including JOE, TAMRA, ROX, FAM, Fluorescein or other moieties for detection (Ju et al. Proc Natl Acad Sci USA 103(52): 19635-40 (2006)). In addition, 3′-azido-ddNTPs can be incorporated by a DNA polymerase and then dye-labeled by “CLICK” chemistry methods (Seo et al. J Org Chem 68(2): 609-12 (2003)). Additional dye terminators incorporated by reference are those described in the catalog of PerkinElmer, Waltham, Mass. (JOE-ddATP; JOE-ddCTP; JOE-ddGTP; JOE-ddUTP; TAMRA-ddATP; TAMRA-ddCTP; TAMRA-ddGTP; TAMRA-ddUTP; FAM-ddATP; FAM-ddCTP; FAM-ddGTP; FAM-ddUTP; ROX-ddATP; ROX-ddCTP; ROX-ddGTP; ROX-ddUTP; Fluorescein-12-ddATP; Fluorescein-12-ddCTP; Fluorescein-12-ddGTP; Fluorescein-12-ddUTP). Other dye terminators include: ROX-acycloNTP; TAMRA-acycloNTP; R6G-ddNTP; R110-ddNTP; Fl-12-acycloNTP; IRD40-ddNTP; IRD700-ddNTP; IRD700-acycloNTP; Cyanine 3-ddNTP; Cyanine 5-ddNTP; Bodipy TR-ddNTP; Bodipy TMR-ddNTP; Bodipy R6G-ddNTP and Bodipy Fl-ddNTP (Gardner and Jack Nucleic Acids Research 30: 605-613 (2002)).

In order to determine the extent of 3′-modified nucleotide incorporation by archaeon DNA polymerases, the titration assay described by Gardner and Jack (2002) was used. In this assay, the efficiency of incorporation of chain terminators was judged by the size of the reaction products in a polymerization reaction. As the efficiency of chain-terminator incorporation increased, the average reaction product size decreased because polymerization was more often halted by terminator addition. By comparing the amount of terminator required to give the same spectrum of reaction products, the relative efficiency of incorporation of the test compounds with the different polymerases was determined.

Several innovations are exploited in novel combinations in present embodiments of the invention to overcome previously noted limitations in chain terminator incorporation. Modified 3′-ddNTP and modified 3′-O-dNTP terminators were identified that are more efficiently incorporated (see Example 2). Methods have been described to identify additional compounds of this type (Example 3). Such compounds offer a marked advantage over previously tested ddNTPs whose incorporation was disfavored.

The efficient production of chain terminator products is useful for genotyping and DNA sequence determination. These methods require traditional chain terminator sequencing, and automated procedures where detection is via incorporation of dye-labeled terminators. Furthermore, reversible terminators allow massively parallel sequencing-by-synthesis strategies. The present invention is applicable to both long-range DNA sequence determination where hundreds of base pairs of contiguous sequence are revealed and to short-range sequencing, defined as little as one base pair of sequence. In the case of short-range sequencing, the present invention is useful in analyzing sequence polymorphisms, for example in genetic testing and screening for specific single nucleotide polymorphisms (SNPs).

All references cited herein, including U.S. provisional application Ser. No. 61/046,987 filed Apr. 22, 2008, are hereby incorporated by reference.

### EXAMPLES

### Example 1

### Titration Assay for Chain Terminator Incorporation Efficiency

Nucleotides: dCTP, ddCTP and acycloCTP were from NEB (Ipswich, Mass.). 3′-amino-ddCTP and 3′-azido-ddCTP were purchased from TriLink Biotech (San Diego, Calif.).

In order to select DNA polymerase mutants with increased reversible terminator incorporation efficiency, mutations were created in 9° N DNA polymerase (Southworth et al. Proc. Natl. Acad. Sci. USA 93: 5281-5285 (1996)) active site residues residing in Region II and Region III (FIGS. 1, 5, and 6).

A complex library of 9° N DNA polymerase mutants was screened for enhanced incorporation of 3′-azido-ddCTP. Several classes of 9° N DNA polymerase mutants were identified with enhanced terminator incorporation. 9° N exo-L408S/Y409A/P410V (Therminator III™, NEB, Inc., Ipswich, Mass.) was purified and characterized in more detail. 9° N DNA polymerase single mutants P410V, L408S, and Y409A and double mutants L408S/P410V, L408S/Y409A, and Y409A/P410V were also purified for comparison. Purification and characterization of DNA polymerase mutants was as described in Gardner and Jack, Nucleic Acids Res. 27:2545-2553 (1999).

In order to compare relative modified nucleotide analog incorporation efficiency, DNA polymerase mutants were tested (FIG. 1) using a titration assay as described by Gardner and Jack (2002). Although the titration assay for chain terminator incorporation efficiency was originally developed to compare incorporation efficiency of ddNTPs, it was here used to monitor incorporation of 3′-modified nucleotides (FIG. 1).

A dye-labeled oligonucleotide primer 5′-CACGACGTTGTAAAACGAC-3′ (SEQ ID NO. 20) was annealed to a single-stranded M13mp18 DNA template (Accession No. X02513) and extended by a DNA polymerase in the presence of various ratios of modified nucleotide:dNTP (10:1, 2.5:1, 1:2.5 or dNTP (no terminator)) in 1×ThermoPol buffer (20 mM Tris-HCl, 10 mM (NH₄)₂SO₄,10 mM KCl, 2 mM MgSO₄, 0.1% Triton X-100, pH 8.8 at 25° C.). Reactions were incubated and termination products were resolved on 20% denaturing polyacrylamide gel electrophoresis (PAGE).

Once the spectrum of termination products was determined, a comparison of the length, uniformity and clarity of these patterns was used to evaluate incorporation of the terminator. Reaction conditions producing shorter products at a given ratio of terminator to normal nucleotides were defined by improved efficiency of incorporation of terminator by the DNA polymerase. Conversely, when comparisons revealed identical banding patterns at different terminator ratios, the lower ratio identified conditions more favorable to terminator incorporation.

The size distribution of termination products was determined by the relative rates of dNTP and terminator incorporation. These competing reactions utilized the same pool of template and continued until replication was halted, either by incorporation of a terminator or by extension to the end of the template. The incorporation efficiency of two terminators was compared using parallel reactions differing only in the type and concentration of terminator. The relative incorporation efficiency of the two terminators was reflected in the concentrations of terminators in the two reactions. For example, if a first reaction contained 10-fold more terminator than a second reaction to generate the same distribution of terminator fragments, then the first terminator was 10-fold less efficient than that of the second.

A library of 9° N DNA polymerase mutants was constructed by PCR amplification of the polymerase genes using gene-specific primers or codon optimization and gene synthesis Czar et al. Trends Biotechnol 27(2): 63-72 (2009)). The ability to incorporate 3′-modified nucleotide terminators was evaluated by the titration assay for chain terminator incorporation efficiency as described by Gardner and Jack (2002).

### Example 2

### Enhanced Incorporation of 3′-azido-ddCTP by a 9° N DNA Polymerase Mutant

A 5′-dye-labeled oligonucleotide primer 5′-CACGACGTTGTAAAACGAC-3′ (SEQ ID NO. 20) was annealed to a single-stranded M13mp18 DNA template (Accession No. X02513) and extended by a 9° N exo-DNA polymerase mutant in the presence of various ratios of modified nucleotide:dNTP (10:1, 2.5:1, 1:2.5 or dNTP (no terminator)). Using the titration assay as described by Gardner and Jack (2002), the relative 3′-azido-dCTP incorporation efficiency was determined for a series of 9° N mutations (9° N exo-L408S/Y409A/P410V>9° N exo-L408S/P410V>9° N exo-P410V>9° N exo-) (FIG. 1).

### Example 3

### Kinetic Analysis of 3′-azido-ddCTP Incorporation by a 9° N DNA Polymerase Mutant

An IR800-dye-labeled synthetic primer 5′-AGTGAATTCG AGCTCGGTAC CCGGGGATCC TCTAGAGTCG ACCTGCAGGC-3′ (SEQ ID NO:18 was annealed to a template 3′-TCACTTAAGC TCGAGCCATG GGCCCCTAGG AGATCTCAGC TGGACGTCCG GATCCTATAC TAATCCC-5′ (SEQ ID NO:19) and used as a substrate to measure rates of 3′-azido-ddCTP incorporation over a three-minute time course. The results are shown in FIG. 2. Reaction aliquots were sampled at 10, 20, 30, 45, 60, and 180 seconds and analyzed by 20% denaturing PAGE where “S” indicates the unextended primer and “P” the fully extended product corresponding to 3′-azido-ddCMP addition at the +1 position(B). 9° N exo-L408S/Y409A/P410V incorporation of varying concentrations of 3′-azido-ddCTP was measured over a three-minute time course. 9° N exo-L408S/Y409A/P410V was found to incorporate 3′-azido-ddCTP efficiently with almost 100% incorporation after three minutes.

Furthermore, the same assay was used to measure 3′-amino-ddCTP incorporation by 9° N exo-L408S/Y409A/P410V (FIG. 3) and to measure rates of incorporation (FIG. 4). Examples of mutants that increased modified nucleotide incorporation by at least two-fold were:

### 9° N D141A/E143A/P410V

### 9° N D141A/E143A/L408S/P410V

### 9° N D141A/E143A/Y409A/S411T

### 9° N D141A/E143A/L408S/Y409A/P410V

### 9° N D141A/E143A/L408P/Y409A/S411T

### 9° N D141A/E143A/Y409A/P410R/S411T

### 9° N D141A/E143A/L408S/Y409A/P410V/S411T

### 9° N D141A/E143A/L408P/Y409A/P410V/S411T

### 9° N D141A/E143A/N491L/Y494S

### 9° N D141A/E143A/N491V/Y494H

### 9° N D141A/E143A/R406S/L408R

### 9° N D141A/E143A/R406L/L408E

### 9° N D141A/E143A/R406T/L408R

### 9° N D141A/E143A/R406V/L408R

### 9° N D141A/E143A/R406T/L408E

### 9° N D141A/E143A/R406V/L408R

### 9° N D141A/E143A/R406E/L408G

### 9° N D141A/E143A/R406P/L408G

### 9° N D141A/E143A/Y409A/R406V

### 9° N D141A/E143A/Y409A/R406S/L408K

### 9° N D141A/E143A/Y409A/R406S/L408R

### 9° N D141A/E143A/Y409A/R406T/L408K

### 9° N D141A/E143A/Y409A/R406T/L408R

### 9° N D141A/E143A/Y409A/R406H/L408G

### 9° N D141A/E143A/Y409A/R406Y/L408G

### 9° N D141A/E143A/Y409A/R406L/L408G

### 9° N D141A/E143A/Y409A/R406P/L408C

### 9° N D141A/E143A/Y409A/R406S/L408I

### 9° N D141A/E143A/Y409A/R406V/L408Y

### 9° N D141A/E143A/Y409A/R406V/A485L

### 9° N D141A/E143A/N491L/Y494S

### 9° N D141A/E143A/N491V/Y494H

### Mma D153A/E155A/L417S/P419V

### Mma D153A/E155A/L417P/Y418A/S420T

Y409A mutation in 9° N, or a corresponding mutation, can be incorporated to increase 3′-modified nucleotide terminator incorporation when combined with additional mutations as described below.

In general, combining the mutations 9° N D141A/E143A/Y409A with a change in R406 to a nucleophilic amino acid (serine, threonine) and L408 to a basic amino acid (arginine or lysine) resulted in an increased 3′-modified nucleotide terminator incorporation.

In general, combining the mutations 9° N D141A/E143A/Y409A and R406 to hydrophobic amino acid (leucine, isoleucine, valine) and L408 to a small amino acid (glycine or alanine) resulted in an increase 3′-modified nucleotide terminator incorporation.

In general, combining the mutations 9° N D141A/E143A/Y409A and R406 to hydrophobic amino acid (leucine, isoleucine, valine) and L408 to a hydrophobic amino acid (leucine, isoleucine, valine) resulted in an increase 3′-modified nucleotide terminator incorporation.

Additionally, enhancements in nucleotide analog incorporation efficiency could be achieved by adding an additional mutation A485L to the mutants described above.

### 9° N D141A/E143A/P410V/A485L

### 9° N D141A/E143A/L408S/P410V/A485L

### 9° N D141A/E143A/Y409A/S411T/A485L

### 9° N D141A/E143A/L408S/Y409A/P410V/A485L

### 9° N D141A/E143A/L408P/Y409A/S411T/A485L

### 9° N D141A/E143A/Y409A/P410R/S411T/A485L

### 9° N D141A/E143A/L408S/Y409A/P410V/S411T/A485L

### 9° N D141A/E143A/L408P/Y409A/P410V/S411T/A485L

### 9° N D141A/E143A/N491L/Y494S/A485L

### 9° N D141A/E143A/N491V/Y494H/A485L

### 9° N D141A/E143A/R406S/L408R/A485L

### 9° N D141A/E143A/R406L/L408E/A485L

### 9° N D141A/E143A/R406T/L408R/A485L

### 9° N D141A/E143A/R406V/L408R/A485L

### 9° N D141A/E143A/R406T/L408E/A485L

### 9° N D141A/E143A/R406V/L408R/A485L

### 9° N D141A/E143A/R406E/L408G/A485L

### 9° N D141A/E143A/R406P/L408G/A485L

### 9° N D141A/E143A/Y409A/R406S/L408K/A485L

### 9° N D141A/E143A/Y409A/R406S/L408R/A485L

### 9° N D141A/E143A/Y409A/R406T/L408K/A485L

### 9° N D141A/E143A/Y409A/R406T/L408R/A485L

### 9° N D141A/E143A/Y409A/R406H/L408G/A485L

### 9° N D141A/E143A/Y409A/R406Y/L408G/A485L

### 9° N D141A/E143A/Y409A/R406L/L408G/A485L

### 9° N D141A/E143A/Y409A/R406P/L408C/A485L

### 9° N D141A/E143A/Y409A/R406S/L408I/A485L

### 9° N D141A/E143A/Y409A/R406V/L408Y/A485L

### Example 4

### Incorporation of Modified Nucleotide, 3′-O-azidomethyl-dCTP

Incorporation of 3′-O-azidomethyl-dCTP by various DNA polymerases was measured using the titration assay as described in Example 1. For each reaction, a ratio of either 10:1 or 1:1 3′-O-azidomethyl-dCTP:dNTP was used to generate a termination pattern. Each band corresponded to a DNA fragment terminated by a 3′-azido-dCMP. A control reaction lacking terminator (dNTP) which yielded large extension products gave assurance that the DNA polymerase was active, and that termination products resulted from terminator incorporation rather than incomplete polymerization. Reactions with Therminator™ (NEB, Inc., Ipswich, Mass.) and 9° N exo-L408P/Y409A/S411T DNA polymerase were incubated at 72° C. for 30 minutes. Reactions with E. coli Polymerase I (pol I) (NEB, Inc., Ipswich, Mass.), Sequenase™ (USB, Inc., Cleveland, Ohio), Mma exo- and Mma exo-L417P/Y418A/S420T were conducted at 37° C. for 30 mintues. Therminator™ (NEB, Inc., Ipswich, Mass.) DNA polymerase was assayed with a 1:1 ratio of acyCTP as control.

E. coli DNA polymerase I (pol I) (NEB, Inc., Ipswich, Mass.) and Sequenase™ (USB, Inc., Cleveland, Ohio) discriminated against 3′-O-azidomethyl-dCTP and failed to terminate synthesis. 9° N exo-L408P/Y409A/S411T incorporated 3′-O-azidomethyl-dCTP efficiently and generated a termination pattern with 10:1 or 1:1 3′-O-azidomethyl-dCTP:dNTP. Methanococcus maripaiudis DNA polymerase exo-(Mma exo-) discriminated against 3′-O-azidomethyl-dCTP and failed to terminate synthesis. Mma exo-L417P/Y418A/S420T incorporated 3′-O-azidomethyl-dCTP efficiently and generated a termination pattern with 10:1 or 1:1 3′-O-azidomethyl-dCTP:dNTP.

The equivalent mutations in 9° N exo-(L408P/Y409A/S411T) and Mma exo-(L417P/Y418A/S420T) resulted in increased 3′-O-azidomethyl-dCTP incorporation suggesting functional conservation despite differences in optimum temperature.

### Example 5

### Determining Specific Activities of DNA Polymerases

9° N D141A/E143A/L408S/Y409A/P410V and 9° N D141A/E143A/Y409V/A485L DNA polymerases were purified by a method described by Gardner and Jack (Nucleic Acids Res 27(12): 2545-53 (1999)). Specific activities for 9° N D141A/E143A/L408S/Y409A/P410V and 9° N D141A/E143A/Y409V/A485L DNA polymerase were determined by measuring the DNA polymerase activity as well as the protein concentration. Briefly, a primer (5′-CGCCAGGGTTTTCCCAGTCACGAC-3′) (SEQ ID NO:21) was annealed to single-stranded M13mp18 (Accession Number: X02513) in 1×Thermopol Buffer (20 mM Tris-HCl, 10 mM (NH4)2SO4, 10 mM KCl, 2 mM MgSO4, 0.1% Triton X-100, pH 8.8 at 25° C.). 9° N D141A/E143A/L408S/Y409A/P410V and 9° N D141A/E143A/Y409V/A485L DNA polymerase activity was measured using a primed M13 substrate as described in Kong, et al. (J. Biol. Chem. 268:1965-1975 (1993)). DNA polymerase activity was converted to units (one unit was the amount of enzyme that incorporated 10 nmol of dNTP into acid-insoluble material 30 minutes at 75° C.) 9° N D141A/E143A/L408S/Y409A/P410V and 9° N D141A/E143A/Y409V/A485L DNA polymerase protein concentration was determined as described in Bradford Anal Biochem 72: 248-54 ((1976). Specific activity of the DNA polymerase is defined by units/mg protein where a unit is the amount of enzyme that will incorporate 10 nM of dNTP into acid insoluble material.

1. A recombinant protein with DNA polymerase activity, comprising:
an amino acid sequence that has at least 90% amino acid sequence identity with SEQ ID NO:1 wherein one or more amino acids in the recombinant protein are mutated compared with the corresponding wild type protein such that the recombinant protein is capable of (i) incorporating one or more nucleotides into a nucleic acid substrate with a specific activity greater than 200, and (ii) incorporating modified nucleotides with at least two fold greater efficiency than a corresponding wild type DNA polymerase.

2. The recombinant protein according to claim 1, wherein the specific activity is greater than 1000.

3. The recombinant protein according to claim 1, wherein the specific activity is greater than 5000.

4. The recombinant protein according to claim 1, wherein at least one mutation is located in SEQ ID NO:1.

5. The recombinant protein according to claim 1, wherein at least one mutation is located in a conserved region identified as Region III.

6. The recombinant protein according to claim 1, wherein at least one mutation is located in the amino acid sequence outside of SEQ ID NO:1.

7. The recombinant protein according to claim 1, wherein the composition is a 9° N archael polymerase, and the mutated amino acids comprise D141A and E143A and an additional mutation selected from the group consisting of: P410V; S411T; L408S/Y409A/P410V; L408P/Y409A/S411T; P410R/S411T; L408S/Y409A/P410V/S411T; L408P/Y409A/P410V/S411T; N491L/Y494S; N491V/Y494H; R406S/L408R; R406L/L408E; R406T/L408R; R406V/L408R; R406T/L408E; R406V/L408R; R406E/L408G; R406P/L408G; Y409A/R406V; Y409A/R406S/L408K; Y409A/R406S/L408R; Y409A/R406T/L408K; Y409A/R406T/L408R; Y409A/R406H/L408G; Y409A/R406Y/L408G; Y409A/R406L/L408G; Y409A/R406P/L408C; Y409A/R406S/L408I; Y409A/R406V/L408Y; Y409A/R406V/A485L; N491L/Y494S; N491V/Y494H; Y409A/R406 (nucleophilic amino acid)/L408 (basic amino acid); Y409A/R406 (hydrophobic amino acid)/L408 (small amino acid); Y409A/R406/L408 (hydrophobic amino acid)/L408 (small amino acid); P410V/A485L; L408S/P410V/A485L; Y409A/S411T/A485L; L408S/Y409A/P410V/A485L; L408P/Y409A/S411T/A485L; Y409A/P410R/S411T/A485L; L4085/Y409A/P410V/5411T/A485L; L408P/Y409A/P410V/S411T/A485L; N491L/Y494S/A485L; N491V/Y494H/A485L; R406S/L408R/A485L; R406L/L408E/A485L; R406T/L408R/A485L; R406V/L408R/A485L; R406T/L408E/A485L; R406V/L408R/A485L; R406E/L408G/A485L; R406P/L408G/A485L; Y409A/R406S/L408K/A485L; Y409A/R406S/L408R/A485L; Y409A/R406T/L408K/A485L; Y409A/R406T/L408R/A485L; Y409A/R406H/L408G/A485L; Y409A/R406Y/L408G/A485L; Y409A/R406L/L408G/A485L; Y409A/R406P/L408C/A485L; Y409A/R406S/L4081/A485L; Y409A/R406V/L408Y/A485L.

8. The recombinant protein according to claim 1, wherein the composition is a Methanococcus maripaludis (Mma) archaeal polymerase, and the mutated amino acids are selected from D153A/E155A/L417S/P419V and D153A/E155A/L417P/Y418A/S420T.

9. The recombinant protein according to claim 1, wherein the one or more modified nucleotides are selected from 3′ terminators and 3′ reversible terminators.

10. The recombinant protein according to claim 1, wherein N is a nucleoside and the R group on the 3′ position of the ribose is substituted by one of the following: 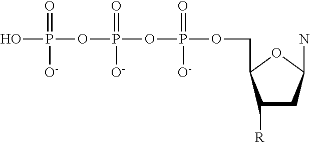
R═—H, —SH, —N₃, —F, —Cl, -azidomethyl, —NH₂, -anthranyloyl -fluothioureido, -chain, -amd, —O-allyl, —O-aminoallyl, —O-azidomethyl,—O-methyl, —O-phophate, —O-diphophate, —O-(2-nitrobenzyl), —O—[N6(anthranyl)amidohex

11. The recombinant protein according to claim 1, wherein N is a nucleoside and the R group on the 3′ position of the ribose is substituted by one of the following: 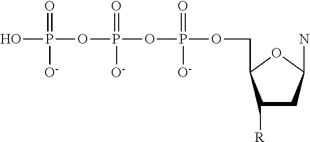
wherein R is larger than a hydroxyl group.

12. The recombinant protein according to claim 10 or 11 wherein R may further comprise a marker.

13. The recombinant protein according to claim 12 wherein the marker is a fluorescent label.

14. The recombinant protein according to claim 1, wherein the modified nucleotides are selected from the group consisting of: 2′-deoxy-3′-anthranyloyl-dNTPs (3′-ant-dNTPs) 3′-{N3-[3-carboxylato-4-(3-oxido-6-oxo-6H-xanthen-9-yl)phenyl]thioureido}-3′-deoxythymidine 5′-triphosphate (3′-fluothioureido-dTTP), 3′-deoxy-3′-(N-methylanthranyloylamino)thymidine 5′-triphosphate (3′-amd-dTTP), 3′-O—[N6(N-methylanthranyl)amidohexanoyl]-dGTP (3-chain-dGTP), and 3′-O—[N6(anthranyl)amidohex (3′-chain-dATP).

15. A method of incorporating modified nucleotides into a nucleic acid, comprising: reacting a nucleic acid with the recombinant protein according to claim 1 and at least one modified nucleotide.

16. A kit comprising the recombinant protein according to claim 1 and instruction for use.

17. A kit according to claim 16, further comprising at least one modified nucleotide.

18. A method of screening for the recombinant protein according to claim 1, comprising:
(a) determining a size of a substrate incorporating a modified nucleotide after a polymerization reaction; and
(b) measuring at least one of an increase in chain-terminator incorporation, and a decrease in average reaction product size, to determine efficiency of incorporation by the composition.


SEQUENCE LISTING (23 sequences)

SEQ ID NO: 1 - protein, 13 aa, Thermococcus sp. 9 degrees N
DFRSLYPSIIITH

SEQ ID NO: 2 - protein, 13 aa, Pyrococcus furiosus
DFRALYPSIIITH

SEQ ID NO: 3 - protein, 13 aa, Thermococcus litoralis
DFRSLYPSIIVTH

SEQ ID NO: 4 - protein, 13 aa, Thermococcus hydrothermalis
DFMSLYPSIIITH

SEQ ID NO: 5 - protein, 775 aa, Thermococcus sp. 9 degrees N
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 6 - protein, 775 aa, Thermococcus sp. 9 degrees N DNAP mutant
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSPAPTIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 7 - protein, 773 aa, Thermococcus gorgonarius
MILDTDYITEDGKPVIRIFKKENGEFKIDYDRNFEPYIYALLKDDSAIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRAIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 8 - protein, 774 aa, Thermococcus kodakarensis
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHGAVIDIYEYDIPFAKRYLIDKGLVPMEGDEELKMLAFDIQTLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITPAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLNYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 9 - protein, 775 aa, Pyrococcus horikoshii
MILDADYITEDGKPIIRIFKKENGEFKVEYDRNFRPYIYALLRDDSAIDEIKKITAQRHGKVVRIVETEKIQRKFLGRPIEVWKLYLEHPQDVPAIRDKIREHPAVVDIFEYDIPFAKRYLIDKGLTPMEGNEKLTFLAVDIETLYHEGEEFGKGPVIMISYADEEGAKVITWKKIDLPYVEVVSSEREMIKRLIRVIKEKDPDVIITYNGDNFDFPYLLKRAEKLGIKLLLGRDNSEPKMQKMGDSLAVEIKGRIHFDLFPVIRRTINLPTYTLEAVYEAIFGKPKEKVYADEIAKAWETGEGLERVAKYSMEDAKVTYELGREFFPMEAQLARLVGQPVWDVSRSSTGNLVEWFLLRKAYERNELAPNKPDEKEYERRLRESYEGGYVKEPEKGLWEGIVSLDFRSLYPSIIITHNVSPDTLNREGCEEYDVAPKVGHRFCKDFPGFIPSLLGQLLEERQKIKKRMKESKDPVEKKLLDYRQRAIKILANSYYGYYGYAKARWYCKECAESVTAWGRQYIDLVRRELEARGFKVLYIDTDGLYATIPGVKDWEEVKRRALEFVDYINSKLPGVLELEYEGFYARGFFVTKKKYALIDEEGKIVTRGLEIVRRDWSEIAKETQARVLEAILKHGNVEEAVKIVKDVTEKLTNYEVPPEKLVIYEQITRPINEYKAIGPHVAVAKRLMARGIKVKPGMVIGYIVLRGDGPISKRAISIEEFDPRKHKYDAEYYIENQVLPAVERILKAFGYKREDLRWQKTKQVGLGAWIKVKKS

SEQ ID NO: 10 - protein, 773 aa, Thermococcus aggregans
MILDTDYITKDGKPIIRIFKKENGEFKIELDPHFQPYIYALLKDDSAIDEIKAIKGERHGKIVRVVDAVKVKKKFLGRDVEVWKLIFEHPQDVPALRGKIREHPAVIDIYEYDIPFAKRYLIDKGLIPMEGDEELKLMAFDIETFYHEGDEFGKGEIIMISYADEEEARVITWKNIDLPYVDVVSNEREMIKRFVQIVREKDPDVLITYNGDNFDLPYLIKRAEKLGVTLLLGRDKEHPEPKIHRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYERNELAPNKPDEEEYRRRLRTTYLGGYVKEPERGLWENIAYLDFSLYPSIIVTHNVSPDTLEREGCKNYDVAPIVGYKFCKDFPGFIPSILGELITMRQEIKKKMKATIDPIEKKMLDYRQRAVKLLANSYYGYMGYPKARWYSKECAESVTAWGRHYIEMTIKEIEEKFGFKVLYADTDGFYATIPGEKPETIKKKAKEFLKYINSKLPGLLELEYEGFYLRGFFVAKKRYAVIDEEGRITTRGLEVVRRDWSEIAKETQAKVLEAILKEDSVEKAVEIVKDVVEEIAKYQVPLEKLVIHEQITKDLSEYKAIGPHVAIAKRLAAKGIKVRPGTIISYIVLRGSGKISDRVILLSEYDPKKHKYDPDYYIENQVLPAVLRILEAFGYRKEDLKYQSSKQVGLDAWLKK

SEQ ID NO: 11 - protein, 774 aa, Thermococcus litoralis
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 12 - protein, 784 aa, Methanococcus maripaludis
MESLIDLDYNSDDLCIYLYLINSIIKEKDFKPYFYVNSTDKEQILEFLKDYEKKHKLDSEISKMIENIETVKKIVFDENYQEKELSKVTVKYPNNVKTVREILMEFERLYEYDIPFVRRYLIDNSVIPTSTWDFENNKKIDNKIPDFKTVSFDIEVYCNKEPNPKKDPIIMASFSSKDFNTVVSTKKFDHEKLEYVKDEKELIKRIIEILKEYDIIYTYNGDNFDFPYLKKRAESFGLELKLGKNDEKIKITKGGMNSKSYIPGRVHIDLYPIARRLLNLTKYRLENVTEALFDVKKVDVGHENIPKMWDNLDETLVEYSHQDAYYTQRIGEQFLPLEIMFSRVVNQSLYDINRMSSSQMVEYLLLKNSYKMGVIAPNRPSGKEYQKRIRSSYEGGYVKEPLKGIHEDIVSMDFLSLYPSIIMSHNLSPETIDCTCCSDEENGENEEILGHKFCKKSIGIIPKTLMDLINRRKKVKKVLREKAEKGEFDEEYQILDYEQRSIKVLANSHYGYLAFPMARWYSRDCAEITTHLGRQYIQKTIEEAENFGFKVIYADTDGFYSKWADDKEKLSKYELLEKTREFLKNINNTLPGEMELEFEGYFKRGIFVTKKKYALIDENEKITVKGLEVVRRDWSNVSKNTQKNVLNALLKEGSVENAKKVIQDTIKELKDGKVNNEDLLIHTQLTKRIEDYKTTAPHVEVAKKILKSGNRVNTGDVISYIITSGNKSISERAEILENAKNYDTNYYIENQILPPVIRLMEALGITKDELKDSKKQYTLHHFLK

SEQ ID NO: 13 - protein, 789 aa, Methanococcus jannaschii
  misc_feature (382)..(382): Xaa can be any naturally occurring amino acid
MGMSMGKIKIDALIDNTYKTIEDKAVIYLYLINSILKDRDFKPYFYVELHKEKVENEDIEKIKEFLLKNDLLKFVENIEVVKKIILRKEKEVIKIIATHPQKVPKLRKIKECEIVKEIYEHDIPFAKRYLIDNEIIPMTYWDFENKKPVSIEIPKLKSVAFDMEVYNRDTEPNPERDPILMASFWDENGGKVITYKEFNHPNIEVVKNEKELIKKIIETLKEYDVIYTYNGDNFDFPYLKARAKIYGIDINLGKDGEELKIKRGGMEYRSYIPGRVHIDLYPISRRLLKLTKYTLEDVVYNLFGIEKLKIPHTKIVDYWANNDKTLIEYSLQDAKYTYKIGKYFFPLEVMFSRIVNQTPFEITRMSSGQMVEYLLMKRAFKXNMIVPNKPDEEEYRRRVLTTYEGGYVKEPEKGMFEDIISMDFRSLYPSIIISYNISPDTLDCECCKDVSEKILGHWFCKKKEGLIPKTLRNLIERRINIKRRMKKMAEIGEINEEYNLLDYEQKSLKILANSVYGYLAFPRARFYSRECAEIVTYLGRKYILETVKEAEKFGFKVLYIDTDGFYAIWKEKISKEELIKKAMEFVEYINSKLPGTMELEFEGYFKRGIFVTKKRYALIDENGRVTVKGLEFVRRDWSNIAKITQRRVLEALLVEGSIEKAKKIIQDVIKDLREKKIKKEDLIIYTQLTKDPKEYKTTAPHVEIAKKLMREGKRIKVGDIIGYIIVKGTKSISERAKLPEEVDIDDIDVNYYIDNQILPPVLRIMEAVGVSKNELKKEGAQLTLDKFFK

SEQ ID NO: 14 - protein, 586 aa, Methanothermobacter thermautotrophicus str. Delta H.
MEDYRMVLLDIDYVTVDEVPVIRLFGKDKSGGNEPIIAHDRSFRPYIYAIPTDLDECLRELEELELEKLEVKEMRDLGRPTEVIRIEFRHPQDVPKIRDRIRDLESVRDIREHDIPFYRRYLIDKSIVPMEELEFQGVEVDSAPSVTTDVRTVEVTGRVQSTGSGAHGLDILSFDIEVRNPHGMPDPEKDEIVMIGVAGNMGYESVISTAGDHLDFVEVVEDERELLERFAEIVIDKKPDILVGYNSDNFDFPYITRRAAILGAELDLGWDGSKIRTMRRGFANATAIKGTVHVDLYPVMRRYMNLDRYTLERVYQELFGEEKIDLPGDRLWEYWDRDELRDELFRYSLDDVVATHRIAEKILPLNLELTRLVGQPLFDISRMATGQQAEWFLVRKAYQYGELVPNKPSQSDFSSRRGRRAVGGYVKEPEKGLHENIVQFDFRSLYPSIIISKNISPDTLTDDEESECYVAPEYGYRFRKSPRGFVPSVIGEILSERVRIKEEMKGSDDPMERKILNVQQEALKRLANTMYGVYGYSRFRWYSMECAEAITAWGRDYIKKTIKTAEEFGFHTVYADTDGFYATYRG

SEQ ID NO: 15 - protein, 903 aa, Bacteriophage RB69
MKEFYLTVEQIGDSIFERYIDSNGRERTREVEYKPSLFAHCPESQATKYFDIYGKPCTRKLFANMRDASQWIKRMEDIGLEALGMDDFKLAYLSDTYNYEIKYDHTKIRVANFDIEVTSPDGFPEPSQAKHPIDAITHYDSIDDRFYVFDLLNSPYGNVEEWSIEIAAKLQEQGGDEVPSEIIDKIIYMPFDNEKELLMEYLNFWQQKTPVILTGWNVESFDIPYVYNRIKNIFGESTAKRLSPHRKTRVKVIENMYGSREIITLFGISVLDYIDLYKKFSFTNQPSYSLDYISEFELNVGKLKYDGPISKLRESNHQRYISYNIIDVYRVLQIDAKRQFINLSLDMGYYAKIQIQSVFSPIKTWDAIIFNSLKEQNKVIPQGRSHPVQPYPGAFVKEPIPNRYKYVMSFDLTSLYPSIIRQVNISPETIAGTFKVAPLHDYINAVAERPSDVYSCSPNGMMYYKDRDGVVPTEITKVFNQRKEHKGYMLAAQRNGEIIKEALHNPNLSVDEPLDVDYRFDFSDEIKEKIKKLSAKSLNEMLFRAQRTEVAGMTAQINRKLLINSLYGALGNVWFRYYDLRNATAITTFGQMALQWIERKVNEYLNEVCGTEGEAFVLYGDTDSIYVSADKIIDKVGESKFRDTNHWVDFLDKFARERMEPAIDRGFREMCEYMNNKQHLMFMDREAIAGPPLGSKGIGGFWTGKKRYALNVWDMEGTRYAEPKLKIMGLETQKSSTPKAVQKALKECIRRMLQEGEESLQEYFKEFEKEFRQLNYISIASVSSANNIAKYDVGGFPGPKCPFHIRGILTYNRAIKGNIDAPQVVEGEKVYVLPLREGNPFGDKCIAWPSGTEITDLIKDDVLHWMDYTVLLEKTFIKPLEGFTSAAKLDYEKKASLFDMFDF

SEQ ID NO: 16 - protein, 898 aa, bacteriophage T4
MKEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFG

SEQ ID NO: 17 - protein, 812 aa, methanoculleus marisnigri JR1
MSVPATLEDFGKIRVGIHQVEYGNVGADTPVVYIFGRDASGKAIQVRVSGFRPYFYAPADLVDGRSLPQEIVGVEENTTYRSIQGVPLRRLYTRRPGDVRAVRDVFSHHYEADIPFTTRFMIDCGLTAGVELPAGAVESFDGAFEIECCELAPAEIKAPARTCIMDIECVDEQGFPEPERDPIICVTCWDSFDDDYTTLLWQPGEAAGDAPDLCVQERHRVVRYPDEIAMLKGLVDYVKKRDPDILSGWNFVEFDIPYIVKRMGALGLKAEDLARIPGQTERNAVRGRSIFDLLGAYRKMHQAQKESYRLDAIAGEELGVTKVRYTGTITDLWRTDPKRLVEYNYRDVELCVGIDQKNNIIEFYREIARYVGCPLDRTLNSSNVIDIFVLRKASGTFVLPSKGLAAGDEFEGATVFEPATGLRENVVVLDLKSLYPMAMMTINASPETKNPDGELRAPNGIRFSREPDGLTRSIIAELLEERDERKRLRNLYPFGSPEYVLYDLQQNVLKVIMNSYYGVSGYTRFRLYDREIGSAVTSVGRAIIRHTRDIITNLGYTVLYGDTDSCMIEVPPGDLEATIARAREIEAKLNASYGDFAKTELNADTHYFSIKFEKVYRRFFQAGKKKRYAGHLVWKEGKDVDEVDVVGFEIRRSDSPQITREVQRAVIEMILRGDAFSDVQAYLRDVIRKYRRGEYSLDEAGIPGGIGKSLDSYENDDAHIRGAKYSNMHLGTDFKRGSKPKRVYIKAVTAKYPRTDVVCFEYADQVPPEFVVDWETMLEKTLKGPLSRIIEPLGWDWHDVDPSRTTLFDFGM

SEQ ID NO: 18 - DNA, 50 bp, artificial
  primer
agtgaattcgagctcggtacccggggatcctctagagtcgacctgcaggc

SEQ ID NO: 19 - DNA, 67 bp, artificial
  template
tcacttaagctcgagccatgggcccctaggagatctcagctggacgtccggatcctatactaatccc

SEQ ID NO: 20 - DNA, 19 bp, artificial
  primer
cacgacgttgtaaaacgac

SEQ ID NO: 21 - DNA, 24 bp, artificial
  primer
cgccagggttttcccagtcacgac

SEQ ID NO: 22 - protein, 784 aa, Methanococcus maripaludis
  MISC_FEATURE (1)..(784): Exo-
MESLIDLDYNSDDLCIYLYLINSIIKEKDFKPYFYVNSTDKEQILEFLKDYEKKHKLDSEISKMIENIETVKKIVFDENYQEKELSKVTVKYPNNVKTVREILMEFERLYEYDIPFVRRYLIDNSVIPTSTWDFENNKKIDNKIPDFKTVSFAIAVYCNKEPNPKKDPIIMASFSSKDFNTVVSTKKFDHEKLEYVKDEKELIKRIIEILKEYDIIYTYNGDNFDFPYLKKRAESFGLELKLGKNDEKIKITKGGMNSKSYIPGRVHIDLYPIARRLLNLTKYRLENVTEALFDVKKVDVGHENIPKMWDNLDETLVEYSHQDAYYTQRIGEQFLPLEIMFSRVVNQSLYDINRMSSSQMVEYLLLKNSYKMGVIAPNRPSGKEYQKRIRSSYEGGYVKEPLKGIHEDIVSMDFLSLYPSIIMSHNLSPETIDCTCCSDEENGENEEILGHKFCKKSIGIIPKTLMDLINRRKKVKKVLREKAEKGEFDEEYQILDYEQRSIKVLANSHYGYLAFPMARWYSRDCAEITTHLGRQYIQKTIEEAENFGFKVIYADTDGFYSKWADDKEKLSKYELLEKTREFLKNINNTLPGEMELEFEGYFKRGIFVTKKKYALIDENEKITVKGLEVVRRDWSNVSKNTQKNVLNALLKEGSVENAKKVIQDTIKELKDGKVNNEDLLIHTQLTKRIEDYKTTAPHVEVAKKILKSGNRVNTGDVISYIITSGNKSISERAEILENAKNYDTNYYIENQILPPVIRLMEALGITKDELKDSKKQYTLHHFLK

SEQ ID NO: 23 - protein, 784 aa, Methanococcus maripaludis
  MISC_FEATURE (1)..(784): exo_ L417P/Y418A/S420T mutant
MESLIDLDYNSDDLCIYLYLINSIIKEKDFKPYFYVNSTDKEQILEFLKDYEKKHKLDSEISKMIENIETVKKIVFDENYQEKELSKVTVKYPNNVKTVREILMEFERLYEYDIPFVRRYLIDNSVIPTSTWDFENNKKIDNKIPDFKTVSFAIAVYCNKEPNPKKDPIIMASFSSKDFNTVVSTKKFDHEKLEYVKDEKELIKRIIEILKEYDIIYTYNGDNFDFPYLKKRAESFGLELKLGKNDEKIKITKGGMNSKSYIPGRVHIDLYPIARRLLNLTKYRLENVTEALFDVKKVDVGHENIPKMWDNLDETLVEYSHQDAYYTQRIGEQFLPLEIMFSRVVNQSLYDINRMSSSQMVEYLLLKNSYKMGVIAPNRPSGKEYQKRIRSSYEGGYVKEPLKGIHEDIVSMDFLSPAPTIIMSHNLSPETIDCTCCSDEENGENEEILGHKFCKKSIGIIPKTLMDLINRRKKVKKVLREKAEKGEFDEEYQILDYEQRSIKVLANSHYGYLAFPMARWYSRDCAEITTHLGRQYIQKTIEEAENFGFKVIYADTDGFYSKWADDKEKLSKYELLEKTREFLKNINNTLPGEMELEFEGYFKRGIFVTKKKYALIDENEKITVKGLEVVRRDWSNVSKNTQKNVLNALLKEGSVENAKKVIQDTIKELKDGKVNNEDLLIHTQLTKRIEDYKTTAPHVEVAKKILKSGNRVNTGDVISYIITSGNKSISERAEILENAKNYDTNYYIENQILPPVIRLMEALGITKDELKDSKKQYTLHHFLK